(19) United States
(12) Patent Application Publication
(10) Pub. No.: US 2026/0043792 A1
(43) Pub. Date: Feb. 12, 2026

(54) METHODS OF IDENTIFYING SELECTIVE CONDENSATE MODULATORS

(71) Applicant: Dewpoint Therapeutics, Inc., Boston, MA (US)

(72) Inventors: John C. MANTEIGA, Boston, MA (US); Drew Cameron MASLAR, Boston, MA (US); Peter Jeffrey DANDLIKER, Boston, MA (US); Nasim JAMALI, Boston, MA (US)

(21) Appl. No.: 19/100,286

(22) PCT Filed: Aug. 2, 2023
(86) PCT No.: PCT/US2023/071541

§ 371 Date: Jan. 31, 2025

Related U.S. Application Data
(60) Provisional application No. 63/370,354, filed on Aug. 3, 2022.

(51) Int. Cl. G01N 33/50

(52) U.S. Cl. CPC G01N 33/5032; G01N 33/5038

(57) ABSTRACT

The present application provides, in some aspects, methods of identifying a stimulus for a condensate modulatory characteristic for one or more condensate types, such as to identify a selective condensate modulator. In other aspects, also provided herein are kits and systems.

### CROSS-REFERENCE TO RELATED APPLICATIONS

This application claims the priority to and benefit of U.S. Provisional Patent Application No. 63/370,354, filed on Aug. 3, 2022, the contents of which are hereby incorporated herein by reference in their entirety.

### TECHNICAL FIELD

The present application relates to the field of biological condensates.

### BACKGROUND

Conventional disease research and application to therapeutic drug discovery focuses on identifying individual biomolecules (molecular targets) that cause or mediate disease-relevant biology, such as via known cellular pathways. This “single target” approach to disease research and therapeutic drug discovery has significant limitations as in vivo systems are extraordinarily complex and our understanding of cellular pathways and biomolecular actions and interactions are often incomplete. Moreover, not all individual biomolecules, when viewed singularly, are suitable drug targets. Such challenges have contributed to the current shortage of new therapeutic drugs to treat unmet medical needs. In the search new therapeutic drugs, biological condensates are currently being evaluated as a drug discovery target and/or tool. Certain initial studies in the field of biological condensates observed chemical entities capable of indiscriminately dissolving and/or preventing the formation of biological condensates. However, chemical entities with such a broad impact may lead to undesirable drug toxicity and off-target properties. There remains a need in the art for new ways to identify therapeutic candidates that selective modulate condensate types and/or condensate components.

### BRIEF SUMMARY

In certain aspects, provided herein is a method of identifying a stimulus for a condensate modulatory characteristic for one or more condensate types, the method comprising: (a) subjecting a cellular composition comprising a cell type to the stimulus, (b) measuring a feature of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker; and (d) identifying the stimulus for the condensate modulatory characteristic from the marker perturbation score.

In some embodiments, the method further comprises the step of: determining a global condensate perturbation score, wherein the global condensate perturbation score is based on at least one marker perturbation score; and the identifying the stimulus for the condensate modulatory characteristic step can be identified from at least one marker perturbation score and/or global condensate perturbation score.

In some embodiments, the measuring the feature step measures two markers for each of the at least one or more condensate types in at least a portion of the stimulus-subjected cellular composition. In some embodiments, the measuring the feature step measures at least one marker for at least two or more condensate types.

In some embodiments, each marker is independently a lipid, a polypeptide, or a nucleic acid. In some embodiments, each marker is independently a polypeptide. In some embodiments, each marker: (i) is within the condensate type; (ii) partitions into the condensate type after subjecting the cellular composition to the stimulus; or (iii) is excluded from the condensate type after subjecting the cellular composition to the stimulus. In some embodiments, each marker is independently a condensate scaffold polypeptide or nucleic acid. In some embodiments, at least one marker is a condensate scaffold polypeptide. In some embodiments, each marker is independently a condensate client polypeptide or nucleic acid.

In some embodiments, the measuring the feature of the at least one marker in at least a portion of the stimulus-subjected cellular composition comprises staining at least a portion of the cellular composition for the marker. In some embodiments, the staining is immunofluorescent (IF) staining.

In some embodiments, the measuring, for at least the portion of the cellular composition, the feature of the at least one marker comprises imaging at least the portion of the cellular composition. In some embodiments, the imaging comprises a fluorescent imaging technique.

In some embodiments, at least one of one or more different condensate types form in the cellular compositions after being subjected the stimulus.

In some embodiments, the stimulus is selected from the group consisting of an exogenous compound, exogenous peptidic material, exogenous genetic material, stressors, an environmental stimulus, and combinations thereof. In some embodiments, the stimulus is an exogenous compound. In some embodiments, the compound is a small molecule therapeutic candidate, or precursor thereof.

In some embodiments, the cellular composition is subjected to the compound at a known concentration.

In some embodiments, the method further comprises subjecting the cellular composition to a second stimulus, measuring a feature of the at least one marker in at least a portion of the second stimulus-subjected cellular composition, and independently determining a marker perturbation score for each marker in the second stimulus-subjected cellular composition based on the measured feature of each marker in the second stimulus-subjected cellular composition.

In some embodiments, the method further comprises comparing a marker perturbation score for a marker in the stimulus-subjected cellular composition with a marker perturbation score for a marker in the second stimulus-subjected cellular composition.

In some embodiments, the method further comprises determining a second global condensate perturbation score to evaluate the second stimulus for the condensate modulatory characteristic, wherein the second global condensate perturbation score is based on at least one marker perturbation score for a marker in the second stimulus-subjected cellular composition.

In some embodiments, the method further comprises comparing the global condensate perturbation score with the second global condensate perturbation score.

In some embodiments, the method further comprises determining at least one characteristic associated with at least one of the one or more condensate types and/or the markers. In some embodiments, the at least one characteristic associated with the at least one of the one or more condensate types and/or the markers comprises: (i) location of the condensate type; (ii) distribution of the condensate type and/or the marker; (iii) number of the condensate type; (iv) size of the condensate type; (v) ratio of the amount of the condensate type and a control condensate; (vi) a functional activity associated with the condensate type; (vii) composition of the condensate type; (viii) co-localization of the condensate type with a biomolecule; (ix) diffusion coefficient of a component of the condensate type; (x) stability of the condensate type; (xi) dissolution or reduction in size of the condensate type; (xii) surface area of the condensate type; (xiii) sphericity of the condensate type; (xiv) liquidity of the condensate type; (xv) solidification of the condensate type; (xvi) location of the marker; (xvii) amount of the marker or a precursor thereof; (xviii) condensate partitioning of the marker into the condensate type; (xix) a functional activity associated with the marker; (xx) aggregation of the marker; (xxi) post-translational modification status of the marker; and (xxii) amount of a degradation product of the marker.

In some embodiments, at least one of the one or more condensate types is selected from the group consisting of a cleavage body, p-granule, histone locus body, a multivesicular body, neuronal RNA granule, nuclear gem, nuclear pore complex, nuclear speckle, nuclear stress body, nucleolus, Oct1/PTF/transcription (OPT) domain, paraspeckle, perinucleolar compartment, PML nuclear body, PML oncogenic domain, polycomb body, processing body, Sam68 nuclear body, stress granule, splicing speckle, P62 body, Cajal body, heterochromatin, transcriptional condensate, and centrosome

In some embodiments, the method comprises measuring a feature of a second marker in at least a portion of the stimulus-subjected cellular composition, wherein the second marker associates with at least one of the one or more condensate types prior to and/or after the cellular composition is subjected to the stimulus. In some embodiments, the method further comprises determining a second marker perturbation score for the second marker in the stimulus-subjected cellular composition based on the measured feature of the second marker. In some embodiments, the marker perturbation score is indicative of whether the stimulus modulates association of the marker with the at least one of the one or more condensate types in the cellular composition, and wherein the second marker perturbation score is indicative of whether the stimulus modulates association of the second marker with the at least one of the one or more condensate types in the cellular composition. In some embodiments, the marker and the second marker associate with the same condensate type. In some embodiments, the marker and the second marker associate with the different condensate types.

In some embodiments, each marker perturbation score is independently based on at least one characteristic associated with at least one of the one or more condensate types and/or the markers.

In some embodiments, each marker perturbation score is independently based on a coefficient of variation (CV) for the marker, and wherein the CV is determined based on a standard deviation (STD) of distribution intensity for the marker divided by the mean distribution intensity of the marker. In some embodiments, the distribution intensity for each marker is based on a pixel analysis of one or more images of the one or more cellular compositions, or a portion of each thereof. In some embodiments, each marker perturbation score is based on a median absolute deviation (MAD) Z-score of the CV for the marker.

In some embodiments, the method further comprises obtaining the MAD Z-score for the marker. In some embodiments. (i) the marker perturbation score is 1 when the MAD Z-score is >5 or <−5; (ii) the marker perturbation score is 0.5 when 2.5 <MAD Z-score≤5 or −5≤MAD Z-score<−2.5; and (iii) the marker perturbation score is 0 when −2.5≤MAD Z-score≤2.5.

In some embodiments, the determining the marker perturbation score for a marker comprises extracting one or more features from an image of the stimulus-subject cellular composition. In some embodiments, the one or more features are based on one or more of: one or more texture features, one or more intensity features, or one or more morphology features. In some embodiments, the determining the marker perturbation score for a marker comprises measuring a plurality of features associated with the marker, calculating a modified Z-score for each feature of each marker based on the measured plurality of features, calculating a feature-change score for each feature of each marker based on the associated modified Z-score and a proportional scale, and then aggregating feature-change scores of a top percentage of the feature-change scores. In some embodiments, the aggregating the modified Z-scores comprises summing or averaging.

In some embodiments, the method further comprises evaluating a reference stimulus, wherein the method comprises determining a reference marker perturbation score. In some embodiments, the reference marker perturbation score is 0, and wherein the marker perturbation score of >0 is indicative of the stimulus having a condensate modulatory characteristic.

In some embodiments, the global condensate perturbation score is indicative of the stimulus selectively modulating the one or more condensate types for the stimulus-subjected cellular composition.

In some embodiments, the global condensate perturbation score is indicative of the stimulus non-selectively modulating the one or more condensate types for the stimulus-treated cellular composition.

In some embodiments, the global condensate perturbation score is indicative of the stimulus not substantially modulating the one or more target condensate types for the stimulus-treated cellular composition.

In some embodiments, the global condensate perturbation score is based on the marker perturbation score and the second marker perturbation score.

In some embodiments, the global condensate perturbation score is calculated by dividing [a sum of all marker perturbation scores] with [the number of marker perturbation scores]. In some embodiments, the global condensate perturbation score is calculated by summing all marker perturbation scores.

In some embodiments, the method comprises determining a reference global condensate perturbation score. In some embodiments, the reference global condensate perturbation score is 1, and wherein: (i) the global condensate perturbation score of 0 <and <1 is indicative of the stimulus selectively modulating the one or more condensate types in the cellular composition; and (ii) the global condensate perturbation score of 0 is indicative of the stimulus not substantially modulating the one or more condensate types in the cellular composition. In some embodiments, for the global condensate perturbation score of 0 <and <1, the smaller the global condensate perturbation score for the stimulus the higher the selectivity of the stimulus.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

All references cited herein, including patent applications and publications, are incorporated herein by reference in their entirety.

### BRIEF DESCRIPTION OF THE DRAWINGS

FIG. 1A and 1B show example cell images having a low coefficient of variation (CV; FIG. 1A) and a high CV (FIG. 1B).

FIGS. 2A and 2B show a visualization of marker perturbation scores and global condensate perturbation scores for 60 candidate compounds at 4 μM concentration which are assessed across 12 condensate types using two markers for each condensate type. Condensate types are provided alphabetically, e.g., A, B, C, etc., condensate components are provided under the condensate type using roman numerals, and compounds are provided numerically, e.g., 1, 2, 3, etc. Global condensate perturbation scores are provided following the compound identifier.

FIGS. 3A and 3B show visualization of marker perturbation scores and global condensate perturbation scores for 40 candidate compounds assessed across 11 condensate types using two markers for each condensate type (22 condensate markers). Condensate components (as identified using markers) are noted using roman numerals located at the column headers. Each row represents a candidate compound, and the global condensate perturbation scores are provided on the left side of each row.

FIGS. 4A and 4B show visualization of marker perturbation scores and global condensate perturbation scores for 40 candidate compounds assessed across 11 condensate types using two markers for each condensate type (22 condensate markers). Condensate components (as identified using markers) are noted using roman numerals located at the column headers. Each row represents a candidate compound, and the global condensate perturbation scores are provided on the left side of each row.

### DETAILED DESCRIPTION

The present application provides, in some aspects, methods of identifying a stimulus for a condensate modulatory characteristic for one or more condensate types, such as to, e.g., identify a small molecule therapeutic candidate that is a selective condensate modulator. In certain aspects, the methods provided herein are useful for identifying a stimulus (e.g., a small molecule therapeutic candidate, including the small molecule therapeutic at a set concentration or range thereof) that modulates a desired condensate type (or a subset of condensate types), including modulation of the condensate(s) in a desired manner. The present disclosure is based, at least in part, on the inventors' findings regarding methodology that enable the identification of selective condensate modulators. In certain aspects, the methods utilizes a marker perturbation score. In certain aspects, the methods further utilizes a global condensate perturbation score. The marker perturbation score and the global condensate perturbation score provide robust and reproducible parameters for identifying selective condensate modulators (with selectivity capable of being assessed at the level of one or more condensate components in a condensate type and/or at the level of one or more condensate types). Additionally, the methods taught herein are amenable to high throughput and commercial-scale drug discovery workflows. These findings, and the methods taught herein, support new ways to more efficiently evaluate biological condensates and modulation thereof useful for therapeutic treatments.

Thus, in some aspects, provided herein is a method of identifying a stimulus for a condensate modulatory characteristic for one or more condensate types, the method comprising: (a) subjecting a cellular composition comprising a cell type to the stimulus, (b) measuring a feature of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker, and (d) identifying the stimulus for the condensate modulatory characteristic from the marker perturbation score.

In some aspects, provided herein is a method of identifying a stimulus for a condensate modulatory characteristic for one or more condensate types, the method comprising: (a) subjecting a cellular composition comprising a cell type to the stimulus, (b) measuring a feature of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker; (d) determining a global condensate perturbation score, wherein the global condensate perturbation score is based on at least one marker perturbation score; and (e) identifying the stimulus for the condensate modulatory characteristic step can be identified from at least one marker perturbation score and/or global condensate perturbation score.

In some aspects, provided herein are systems useful for performing at least certain aspects of the methods provided herein, such as for computer-implementation of aspects of the methods provided herein. In some embodiments, the system is configured to perform measuring a feature of at least one marker in at least a portion of a stimulus-subjected cellular composition. In some embodiments, the system is configured to perform determining a marker perturbation score. In some embodiments, the system is configured to perform determining a global condensate perturbation score. In some embodiments, the system comprises a user-interface, such as a screen, touchpad, and/or buttons or keys for providing commands and/or reviewing information related to the computer-implementation of the methods provided herein.

In some aspects, provided herein are compositions, such as kits, useful for the methods provided herein. In some embodiments, provided herein is a kit comprising one or more antibodies that recognize one or more condensate components, e.g., a marker panel.

The section headings used herein are for organizational purposes only and are not to be construed as limiting the subject matter described. For example, some aspects of the disclosure are presented in a modular fashion, and such presentation is not to be construed as limited the possible combinations of approaches taught herein.

### I. DEFINITION

For purposes of interpreting this specification, the following definitions will apply and. whenever appropriate, terms used in the singular will also include the plural and vice versa. In the event that any definition set forth below conflicts with any document incorporated herein by reference, the definition set forth shall control.

As used herein, “condensate” means a non-membrane-encapsulated compartment formed by phase separation of one or more proteins and/or other macromolecules such as nucleic acids (including all stages of phase separation).

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, including but not limited to, methylation, phosphorylation glycosylation, sialylation, or acetylation.

The term “antibody,” and grammatical equivalents thereof, includes full-length antibodies and antigen-binding fragments thereof. A full-length antibody comprises two heavy chains and two light chains. The term “antigen-binding fragment” as used herein refers to an antibody fragment including, for example, a diabody, a Fab, a Fab', a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multi-specific antibody formed from a portion of an antibody comprising one or more CDRs, a camelized single domain antibody, a nanobody, a domain antibody, a bivalent domain antibody, any other antibody fragment that binds to an antigen but does not comprise a complete full-length antibody structure, or an antibody mimetic (e.g., designed ankyrin repeat proteins (DARPin), affimer, or monobody (ADNECTINS®)). An antigen-binding fragment is capable of binding to the same antigen to which the parent antibody or a parent antibody fragment (e.g., a parent scFv) binds.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. METHODS OF IDENTIFYING AND/OR ASSESSING CONDENSATE MODULATORS

Provided herein, in certain aspects, are methods of identifying a stimulus associated with a condensate modulatory characteristic, such as a stimulus that produces or contributes to a change to a characteristic of a condensate or a component thereof. In some embodiments, the methods taught herein can be used to assess one or more stimuli for a one or more condensate modulatory characteristics, including for determining that a stimulus is not associated with a condensate modulatory characteristic. In some embodiments, the methods taught herein can be used to identify one or more stimuli having a selective effect on one or more condensate types and/or one or more condensate components.

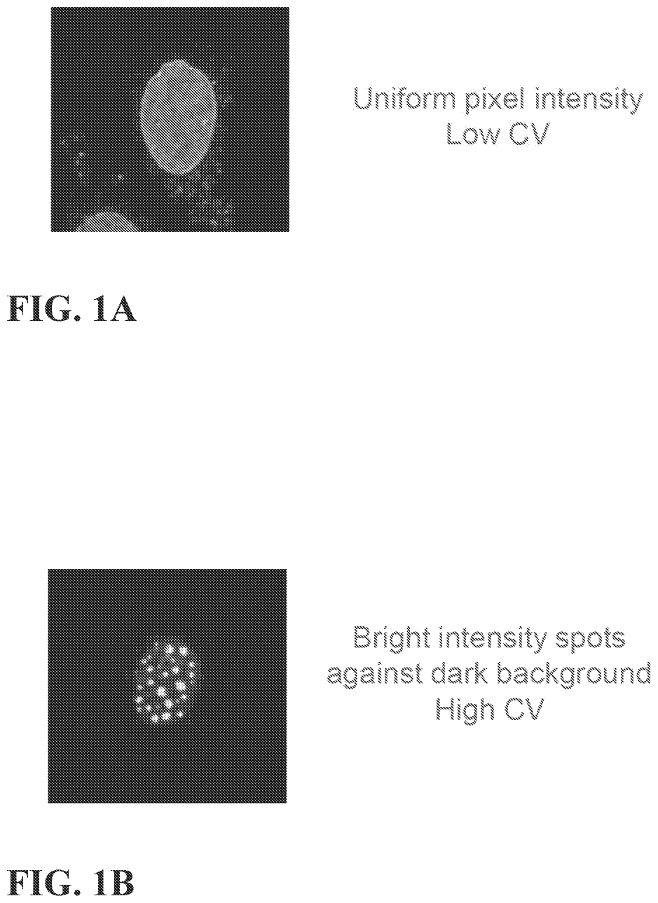

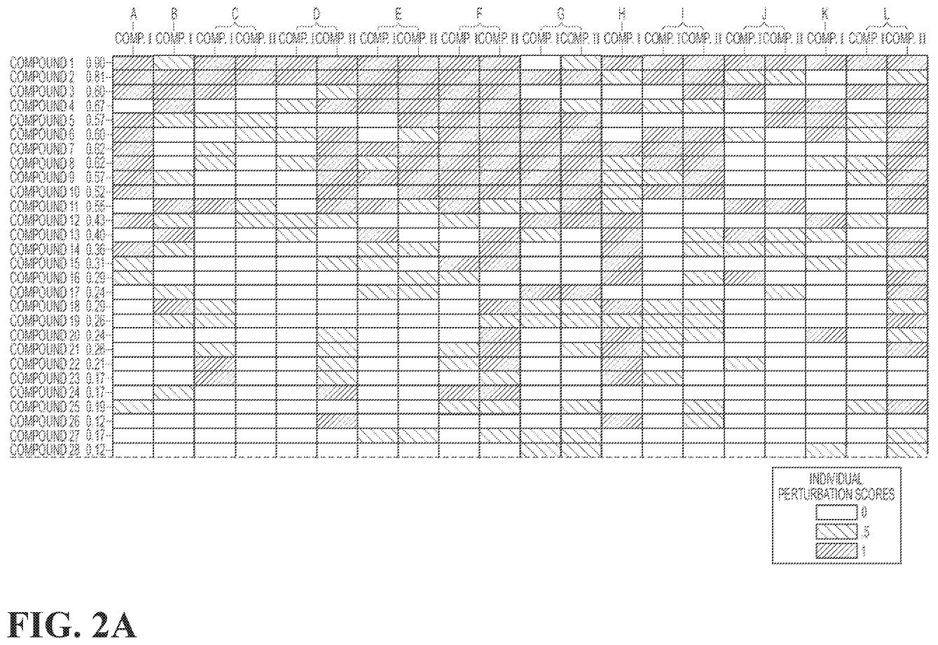

As discussed in more detail herein, in some embodiments, the methods provided herein involve a marker perturbation score. In some embodiments, exposing a cellular composition comprising a cell to a stimulus may result in a change to a condensate or a component thereof, and such change can be measured using a known marker of said condensate or component thereof (e.g., a polypeptide that is associated with the condensate and/or associates or disassociates with the condensate under certain conditions, such as in response to a stimulus). In some embodiments, the marker is, or associates with, a condensate component thereby enabling detection of the condensate component, whether or not the condensate component is associated with a condensate type. Accordingly, in some embodiments, the marker perturbation score is based on alterations (or a lack thereof) in the feature of a marker for a condensate type following exposure to a stimulus, which may be indicative of the stimulus having an effect on the condensate or a component thereof. For example, after a cellular composition comprising a cell type is exposed to a stimulus, the feature of a condensate component known to associate with a condensate type can be assessed for at least a portion of the cellular composition, such as a field of view (or a portion thereof) of a cell therein. In some embodiments, the feature, such as a distribution, is assessed using a coefficient of variation (CV) calculation, such as determined via the standard deviation of a signal attributable to a marker in an area divided by the mean signal attributable to a marker in the area. In some embodiments, the feature is assessed using an index of dispersion. As illustrated in FIG. 1A, for a scenario with uniform intensity, such as based on pixels, in an area (here, the nucleus of a cell), the coefficient of variation is low as the standard deviation of the signal is low relative to the mean signal in the area. As illustrated in FIG. 1B, for a scenario with bright intensity spots (e.g., due to the marker being in a condensate) and a dark background in an area (here, the nucleus of a cell), the coefficient of variation is high as the standard deviation of the signal is high relative to the mean signal in the area.

In some embodiments, the methods provided herein involve a global condensate score. In some embodiments, the global condensate score is a metric useful for assessing the selectivity of a stimulus across one or more condensate types. In some embodiments, the global condensate perturbation score is based on a Z-score, such as a median-based Z-score.

Thus, in certain aspects, provided is a method of identifying a stimulus for a condensate modulatory characteristic for one or more condensate types, such as one or more condensate types in a specific cell type, the method comprising. (a) subjecting a cellular composition comprising a cell type to the stimulus, (b) measuring a feature of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker; and (d) identifying the stimulus for the condensate modulatory characteristic from a marker perturbation score.

In certain aspects, provided is a method of identifying a stimulus for a condensate modulatory characteristic for one or more condensate types, the method comprising: (a) subjecting a cellular composition comprising a cell type to the stimulus, (b) measuring a feature of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker; (d) determining a global condensate perturbation score, wherein the global condensate perturbation score is based on at least one marker perturbation score, and (e) identifying the stimulus for the condensate modulatory characteristic from a marker perturbation score, wherein the identifying the stimulus for the condensate modulatory characteristic step can be identified from at least one marker perturbation score and/or a global condensate perturbation score.

Methodology and elements of assays involving marker perturbation scores and, optionally, global condensate scores are described in more detail throughout the application, including the sections below. The modular discussion of such methodology and elements is not intended to limit the scope of the teachings provided herein, and one of ordinary skill in the art will readily appreciate combinations thereof in light of the description provided herein.

### A. Cellular Compositions, Components Thereof, and Stimuli

In certain aspects, the methods provided herein involve one or more cellular compositions, components thereof, and one or more stimuli. For example, in some embodiments, the method comprises subjecting a cellular composition comprising a cell type to a stimulus.

### 1. Cellular Compositions

In certain aspects, the methods described herein involve one or more cellular compositions. The cellular compositions may comprise any cell type or any mixture of cell types.

In some embodiments, the cellular composition comprises at least about 1,000 cells, such as at least about any of 1,000 cells, 5,000 cells, 1×10^{∝}cells, 5×10⁴, 1×10⁵, 5×10⁵, or 1×10⁶ cells of a cell type.

In some embodiments, the cellular composition comprises a single cell type. In some embodiments, the cellular composition comprises at least about 1,000 cells of a single cell type. In some embodiments, the cellular composition comprises a plurality of cell types, such as any of 2, 3, 4, or 5 cell types. In some embodiments, the cellular composition is aliquoted for use in the methods described herein, such as aliquots containing at least about 100 cells, such as at least about any of 200 cells, 300 cells, 400 cells, 500 cells, 600 cells, 700 cells, 800 cells, 900 cells, 1,000 cells, 1250 cells, 1,500 cells, 1,750 cells, or 2,000 cells.

In some embodiments, the cellular composition comprises cells at or exceeding a desired confluency. For example, in some embodiments, the cellular composition comprises cells at (including grown to) a confluency of at least about 60%, such as at least about any of 65%, 70%, 75%, 80%, 85%, 90%, 95%, or 100%.

In some embodiments, the cellular composition comprises a cell type from an animal, such as a human, rat, or mouse. In some embodiments, the cell type from the animal has one or more features of a disease, such as a neurodegenerative, proliferative, immunological, cardiac, infection, or metabolic disease. In some embodiments, the cellular composition is useful for studying a disease or an aspect or factor thereof. In some embodiments, the cellular composition comprises a HeLa cell, a HEK293 cell, a DLD1 cell, a U2OS cell, a H9C2 cell, an induced pluripotent stem cell (iPSC cell), a cardiomyocyte, a myocyte, a stem cell-derived cell, a neuron, a cancer cell, an immune cell, or an adipocyte. In some embodiments, the cell type is derived from a biopsy or tissue sample, such as from a patient sample, e.g., from a healthy or disease biopsy or tissue sample. In some embodiments, the cellular composition is a tissue sample, such as a tissue sample, cell smear, or secretion. When describing a cell type, it is understood to include cells derived from that type of cell unless explicitly stated otherwise. For example, a HeLa cell comprising a heterologous transgene would be considered a HeLa cell unless explicitly stated otherwise. In some embodiments, the cellular composition comprises a cell type that is modified, such as stably or transiently transfected.

In some embodiments, the cellular composition is a cultured cellular composition, such as a cellular composition contained in a sample well or culture dish. In some embodiments, the method involves a plurality of aliquot of a cellular composition, such as a cellular composition aliquoted in two or more wells of a multi-well plate, e.g., a 1536-well plate.

In other aspects, provided herein are methods comprising identifying, obtaining, and/or making a cellular composition. In some embodiments, the method comprises producing a cellular composition having a cell type. In some embodiments, the cell type of a cellular composition is treated and/or engineered to adjust (such as increase or decrease) the expression of one or more polypeptides. Techniques for making cellular compositions are well known in the art. In some embodiments, the cell type is produced from a precursor cell of a cell type via modulating an aspect of the precursor. For example, the cell type can be generated by subjecting a precursor cell to stress, such as oxidative stress, or treating a precursor cell with a small molecule compound or hormone. In some embodiments, the cell type is obtained by subjecting a precursor cell to infection, such as an infection by virus, bacteria, fungus, or parasite. In some embodiments, the cell type is produced via knock-down or knock-out of a genetic feature or expression product thereof, such as by any methods known in the art, e.g., siRNA, RNAi, TALEN, ZFN, or CRISPR/Cas. In some embodiments, the cell type is produced via knock-in. In some embodiments, the cell type is produced via transfection. In some embodiments, the cell type is transfected with a fusion polypeptide, such as a polypeptide fused to a label, e.g., GFP. In some embodiments, the cell type is transfected with a wild type polypeptide. In some embodiments, the cell type is transfected with a variant polypeptide, such as a mutant polypeptide. In some embodiments, the cell type is transfected to express a level of a gene expression product. In some embodiments, the expression level variant cell type is produced and used in the methods described herein when a gene expression product reaches a pre-determined level

In some embodiments, the cell type is transfected to express a polypeptide, such as a wild type polypeptide or a mutant polypeptide, at a near endogenous level. In some embodiments, the cell type does not express a polypeptide, such as a wild type polypeptide, and the near endogenous level is based on a level of expression of the polypeptide in another cell type. In some embodiments, the cell type is transfected to express a polypeptide with a label, such as a labeled wild type polypeptide, at a near endogenous level, wherein the near endogenous level is based on the level of expression of a respective unlabeled version of the polypeptide. In some embodiments, the cell type has reduced expression of an unlabeled polypeptide, e.g., the cell type comprises a knockout of the unlabeled polypeptide. In some embodiments, the cell type is transfected to express a variant polypeptide, such as a mutant polypeptide (e.g., point mutation, truncation mutation, frameshift mutation, or termination mutation), at a level that is substantially similar to the endogenous expression level of a respective wild type polypeptide of the variant polypeptide. As described herein, in some embodiments, the terms “near endogenous level” or “substantially similar” refer to polypeptide expression levels that are within a 2-fold difference of a measured endogenous level of a polypeptide as measured in a population of cells, such as 1×10⁶ cells.

### 2. Stimuli and Treatments of Cellular Compositions

In certain aspects, the methods provided herein comprise subjecting a cellular composition comprising a cell type to a stimulus. The methods provided herein enable the evaluation of any stimulus. For ease of describing the subject matter provided herein, a cellular composition is generally described as being subjected to a singular stimulus (although this is not a limitation to the description provided herein), and the singular stimulus may encompass numerous factors. For example, a stimulus may be described as a compound, and may also implicitly include any light, heat, O₂ concentration and/or CO₂ concentration factors to which the cellular composition is subjected. In some embodiments, a stimulus is described as a concentration of a compound, wherein two or more cellular composition are subjected to the compound at different concentrations (or, e.g., one concentration of the compound and one vehicle control). In some embodiments, the stimuli are configured such that a compound is evaluated in a manner to evaluate a dose response, e.g., the compound is evaluated at 2 or more, 3 or more, 4 or more, 5 or more, 6 or more, 7 or more, 8 or more, 9 or more, or 10 or more concentrations. One of ordinary skill in the art will readily understand how to evaluate one or more factors of a stimulus by setting up adequate experimental procedures and references.

In some embodiments, the stimulus comprises an exogenous compound. In some embodiments, the stimulus comprises an exogenous peptidic material, such as a hormone. In some embodiments, the stimulus comprises an exogenous genetic material. In some embodiments, the stimulus comprises a stressor. In some embodiments, the stimulus comprises an environmental stimulus. In some embodiments, the stimulus comprises any combination of an exogenous compound, an exogenous peptidic material, an exogenous genetic material, a stressor, or an environmental stimulus.

In some embodiments, the stimulus, such as an exogenous compound, is a small molecule therapeutic candidate or a precursor thereof (e.g., a prodrug). In some embodiments, the stimulus is a known concentration of a small molecule therapeutic candidate. In some embodiments, the stimulus is a mixture of small molecule therapeutic candidates (applied to the cellular composition via a single or multiple compositions). In some embodiments, the stimulus is a small molecule therapeutic candidate and another agent (applied to the cellular composition via a single or multiple compositions). In some embodiments, the stimulus is a polypeptide, a peptidomimetic, a lipid, a nucleic acid, or any combination thereof.
As used herein, small molecule therapeutic candidate encompasses any small molecule being evaluated for purposes of drug discovery, such as would be involved in efforts to identify a therapeutic for a human disease. In some embodiments, the small molecule therapeutic is a member of a small molecule library, such as a chemical compound library. In some embodiments, there may exist some knowledge (obtained before or after performing the methods described herein) that a particular small molecule is not suitable for human therapeutic use—in such instances, said small molecule is still considered a small molecule therapeutic candidate for purposes of this description.

In some embodiments, the small molecule therapeutic candidate has a molecular weight of about 5,000 Da or less, such as about any of 4,500 Da or less, 4,000 Da or less, 3,500 Da or less, 3,000 Da or less, 2,500 Da or less, 2,000 Da or less, 1,500 Da or less, 1,000 Da or less, or 500 Da or less. In some embodiments, the small molecule therapeutic candidate satisfies one or more of Lipinski's rule of five—no more than 5 hydrogen bond donors, no more than 10 hydrogen bond acceptors, a molecular weight of less than 500 Da, and an octanol-water partition coefficient (log P) that does not exceed 5. In some embodiments, the small molecule therapeutic candidate has a desired partitioning characteristic for a condensate type. For example, in some embodiments, the small molecule therapeutic candidate partitions into a condensate type (e.g., the concentration of the small molecule therapeutic candidate is higher in the condensate type than outside the condensate type), such as when it is desired to deliver the small molecule therapeutic candidate to the condensate type. In some embodiments, the small molecule therapeutic candidate does not substantially partition into a condensate type (e.g., the concentration of the small molecule therapeutic candidate is lower in the condensate type than outside the condensate type), such as when it is desired to avoid having the condensate type serve as a sink for the small molecule therapeutic candidate.

In some embodiments, the small molecule therapeutic candidate comprises a nucleic acid. In some embodiments, the small molecule therapeutic candidate comprises RNA, such as a siRNA, miRNA, mRNA, or lnRNA, or an analog thereof. In some embodiments, the small molecule therapeutic candidate comprises DNA, or an analog thereof. In some embodiments, the small molecule therapeutic candidate is a non-naturally occurring compound. In some embodiments, the small molecule therapeutic candidate is an exogenous compound. In some embodiments, the small molecule therapeutic candidate comprises a polypeptide. In some embodiments, the small molecule therapeutic candidate is a therapeutic compound approved by a regulatory agency, such as an agent approved for medical treatment by the United States Food and Drug Administration (FDA). In some embodiments, the small molecule therapeutic candidate is a new chemical entity.

In some embodiments, the small molecule therapeutic candidate, or a portion thereof, is charged. In some embodiments, the small molecule therapeutic candidate, or a portion thereof, is hydrophobic. In some embodiments, the small molecule therapeutic candidate, or a portion thereof, is hydrophilic. In some embodiments, the small molecule therapeutic candidate, or a portion thereof, comprises an alkaloid, a glycoside, a phenazine, a phenol, a polyketide, a terpene, or a tetrapyrrole.

In some embodiments, the small molecule therapeutic candidate comprises a label. In some embodiments, the label is a radioactive label, a colorimetric label, a luminescent label, a chemically-reactive label (such as a component moiety used in click chemistry), or a fluorescent label. In some embodiments, the small molecule therapeutic candidate contains a measurable signal, e.g., has a fluorescent characteristic without further modification. In some embodiments, the small molecule therapeutic candidate comprises a fluorophore. In some embodiments, the small molecule therapeutic candidate is a polypeptide comprising a label. In some embodiments, the small molecule therapeutic candidate is a polypeptide comprising a fluorophore. In some embodiments, the small molecule therapeutic candidate is a nucleic acid comprising a label. In some embodiments, the small molecule therapeutic candidate is a nucleic acid comprising a fluorophore. The label can be associated with the small molecule therapeutic candidate covalently or non-covalently.

In some embodiments, the stimulus is a reference control. For example, in some embodiments wherein a first cellular composition is subjected to a small molecule therapeutic candidate delivered to the first cellular composition in a vehicle, a second cellular composition is subjected to a second stimulus that is a reference control comprising the vehicle. In some embodiments, the reference control stimulus comprises DMSO. In some embodiment, the reference control comprises a cellular composition not contacted with one or more factors of a stimulus, such as a small molecule therapeutic candidate or a vehicle control.

The methods provided herein are amenable to any treatment regimen involving subjecting the cellular composition to a stimulus. In some embodiments, subjecting the cellular composition to a stimulus comprises subjecting the cellular composition to the stimulus in a single instance, such as a heat treatment or application of a chemical composition, such as a small molecule therapeutic candidate. Such single treatments may comprise any length of duration or incubation, e.g., application of a chemical composition for a set time before the cellular composition media is removed and replaced with a media not comprising the chemical composition. In some embodiments, subjecting the cellular composition to a stimulus comprises subjecting the cellular composition to the stimulus for a plurality of instances, such as to reflect chronic exposure or a chronic disease.

### 3. Assay Formats

In certain aspects, the methods provided herein comprise independently subjecting any number of cellular compositions to a stimulus, wherein the methods enable the evaluation of a plurality of stimuli across one or more cell types. In some embodiments, the method comprises performing replicates of subjecting a cellular composition comprising a cell type to a stimulus, e.g., triplicate replicates. The methods described herein can be performed in a diverse array of formats, including formats amenable to high throughput and commercial scale drug discovery.

In some embodiments, the method provided herein comprises aliquoting a cellular composition into a plurality of sample wells, and subjecting each defined subset of the aliquoted cellular composition to a stimulus. In some embodiments, the cellular composition is aliquoted into wells of a multi-well plate, such as a multi-well plate having any of 6, 12, 24, 48, 96, 384, or 1,536 wells. In some embodiments, the multi-well plate is amenable to other steps of the methods described herein, e.g., suitable for the stimulus and any processing and analytical steps performed on the cell type of the cellular composition.

In some embodiments, the method provided herein comprises evaluating a plurality of cellular compositions, wherein each cellular composition comprises a different cell type or derivative thereof. In some embodiments, each cellular composition can be independently subjected to a plurality of stimuli such as via subjecting different aliquots of the cellular composition.

In some embodiments, the number of individual cellular compositions (including aliquots of a cellular composition) used in a method described herein may be determined based on one or more aspects of the desired analysis, including, but not limited to, the number of stimuli to be evaluated, the number of cell types to be evaluated, the number of replicates to be evaluated, and a level of statistical power.

### 4. Post-stimulus Cellular Composition Processing

In some embodiments, the methods provided herein comprise a processing technique performed after subjecting the cellular composition to a stimulus. Such processing techniques are useful for preserving biological context following treatment with a stimulus and prior to certain downstream analyzes (such as staining and/or imaging) and/or preparing the cellular composition or components thereof for certain downstream analyzes.

For example, in some embodiments, the following subjecting a cellular composition comprising a cell type to a stimulus, the cellular composition, or a component thereof such as the cell type, is fixed and/or permeabilized. In some embodiments, the fixing is performed to maintain a cell in a set place, such as accomplished using a crosslinking reagent, e.g., formaldehyde or an analog thereof. In some embodiments, the permeabilizing is performed to enable a marker, such as an antibody, to penetrate a cell, such as accomplished using solvents (e.g., acetone) and/or detergents (e.g., Triton, NP-40, Tween 20, Saponin, Digitonin, and/or Leucoperm). In some embodiments, the following subjecting a cellular composition comprising a cell type to a stimulus, the cellular composition, or a component thereof such as the cell type, is frozen.

### 5. Condensate Types and Condensate Modulatory Characteristics

As discussed herein, the provided methods are useful for identifying a stimulus for a condensate modulatory characteristic for one or more condensate types. Thus, in some embodiments, the cellular composition comprises one or more condensate types. In some embodiments, the cellular composition comprises a condensate type prior to subjecting the cellular composition to a stimulus. In some embodiments, the cellular composition comprises a condensate type after subjecting the cellular composition to a stimulus. In some embodiments, the method further comprises a step of generating a condensate type, such as via gene expression and/or subjecting the cellular composition to a stress. In some embodiments, the cellular composition does not comprise a condensate type, including after subjecting the cellular composition to a stimulus (e.g., a condensate type is observed with subjecting the cellular composition to at least a certain concentration of a small molecule therapeutic candidate).

In certain aspects, the methods described herein involve assessing one or more condensate types, such as via measuring a component thereof. Condensates discussed herein comprise one or more components, such as macromolecules, e.g., a polypeptide and/or nucleic acid such as DNA or RNA. As described herein, a condensate type may be characterized based on the presence of one or more components in the dense phase, such as a specific macromolecule, e.g., a specific polypeptide. In some embodiments, the condensate component is a condensate scaffold. Condensate scaffolds are components that contribute to the structural integrity of a condensate, are often involved in condensate formation, and tend to be more statically present in a condensate. In some embodiments, the condensate component is a condensate client. Condensate clients are components that are not essential for condensate formation, and tend to be present in a condensate under certain conditions. In some embodiments, the condensate type is a known condensate type, e.g., a cleavage body, p-granule, histone locus body, a multivesicular body, neuronal RNA granule, nuclear gem, nuclear pore complex, nuclear speckle, nuclear stress body, nucleolus, Oct1/PTF/transcription (OPT) domain, paraspeckle, perinucleolar compartment, PML nuclear body, PML oncogenic domain, polycomb body, processing body, Sam68 nuclear body, stress granule, splicing speckle, P62 body, Cajal body, heterochromatin, transcriptional condensate, or centrosome. In some embodiments, the condensate type is a centrosome. In some embodiments, the condensate type is a nuclear pore complex. In some embodiments, the condensate type is a P-62 body. In some embodiments, the condensate type is a heterochromatin condensate. In some embodiments, the condensate type is a paraspeckle. In some embodiments, the condensate type is a nucleolus. In some embodiments, the condensate type is a nuclear speckle. In some embodiments, the condensate type is a Cajal body. In some embodiments, the condensate type is a PML body. In some embodiments, the condensate type is a P-body. In some embodiments, the condensate type is a stress granule. In some embodiments, the condensate type is a transcriptional condensate.

In some embodiments, the condensate type is a cellular condensate, such as a condensate existing within a cell type of a cellular composition. In some embodiments, the condensate type is an extracellular condensate, e.g., a condensate type that may exist in an extracellular space of the cellular composition. Extracellular condensates can form in biological solutions outside of a cell, such as the extracellular matrix or plasma, to facilitate reactions or sequester molecules. See, Muiznieks et al., J Mol Biol, 43, 2018, 4741-4753. In some embodiments, the condensate type is a disease-associated condensate type, such as a condensate associated with a disease. In some embodiments, the condensate type is non-naturally occurring. In some embodiments, the condensate type is naturally occurring.

As described herein, a condensate modulatory characteristic refers to a measurable change in one or more characteristics of a condensate or a component thereof, e.g., a marker, that is associated with a stimulus (whether or not that component is associated with the condensate at the time of measurement, e.g., a condensate component that disassociates with a condensate). In some embodiments, the condensate modulatory characteristic of a stimulus is one or more of one or more of: (i) location of the condensate type; (ii) distribution of the condensate type and/or the condensate component; (iii) number of the condensate type; (iv) size of the condensate type: (v) ratio of the amount of the condensate type and a control condensate; (vi) a functional activity associated with the condensate type; (vii) composition of the condensate type; (viii) co-localization of the condensate type with a biomolecule; (ix) diffusion coefficient of a component of the condensate type; (x) stability of the condensate type; (xi) dissolution or reduction in size of the condensate type; (xii) surface area of the condensate type; (xiii) sphericity of the condensate type; (xiv) liquidity of the condensate type; (xv) solidification of the condensate type; (xvi) location of the condensate component; (xvii) amount of the condensate component or a precursor thereof; (xviii) condensate partitioning of the condensate component into the condensate type; (xix) a functional activity associated with the condensate component; (xx) aggregation of the condensate component; (xxi) post-translational modification status of the condensate component; or (xxii) amount of a degradation product of the condensate component. In some embodiments, the condensate modulatory characteristic of a stimulus includes a distribution the condensate type and/or condensate component, such as measured throughout a cell. In some embodiments, the methods provided herein comprise determining at least one characteristic associated with at least one of one or more condensate types and/or condensate component thereof.

In some embodiments, the condensate modulatory characteristic of a stimulus is based on one or more observable or measurable characteristics of a type condensate, which in some aspects herein is referred to as the phenotype of the condensate type or phenotypic identifiers. For example, observable or measurable characteristics or phenotypic identifiers associated with a condensate may be determined by imaging a cellular composition. Observable or measurable characteristics of a condensate phenotype include, but are not limited to, presence (including absence and level/amount), location, distribution, kinetics (such as kinetics of formation or dissolution), morphological (e.g., size, shape, sphericity), material (e.g., fluidity or rigidity), and compositional properties of a condensate. In some embodiments, the condensate phenotype comprises the presence of a condensate type of interest. In some embodiments, the condensate phenotype comprises the absence (including disappearance or dissolution) of a condensate type of interest. In some embodiments, the condensate phenotype comprises the amount of a condensate type of interest, including amount based on number of individual condensates and/or a size feature. In some embodiments, the condensate phenotype comprises the amount of a condensate type of interest comprising and/or not comprising a component (e.g., marker such as biological marker, or one or more other biomolecules that become components of the condensate under certain conditions). In some embodiments, the condensate phenotype comprises the abundance (or level of association) of a component of the condensate type of interest within the condensate type of interest. In some embodiments, the condensate phenotype comprises the location of a condensate type of interest or component thereof, such as the subcellular location. In some embodiments, the condensate phenotype comprises the distribution of a condensate type of interest or component thereof (e.g., relative to other cellular organelles, other condensates, or other biomolecules). In some embodiments, the condensate phenotype comprises a morphological feature of a condensate type of interest in a cell model, such as size, shape, volume, surface area, and/or sphericity. In some embodiments, the condensate phenotype comprises the number of a condensates type per cell, or a region therein. In some embodiments, the condensate phenotype comprises the composition of a condensate type of interest. In some embodiments, the condensate phenotype comprises the behavior or material property of a condensate type of interest, such as dynamic property, liquidity, solidity, or fiber formation. In some embodiments, the condensate phenotype comprises information regarding the kinetics of condensate formation. In some embodiments, the condensate phenotype comprises information regarding the kinetics of condensate dissolution. In some embodiments, the condensate phenotype comprises changes in a phenotypic identifier, such as a formation or dissolution characteristic, in response to an external stimulus.

### 6. Condensate Formation

As discussed herein, in some embodiments, the methods provided herein comprise a step of condensate formation. In some embodiments, the method comprises subjecting a cellular composition to a condition that promotes the formation of a condensate type. In some embodiments, the step of subjecting a cellular composition to a condition that promotes the formation of a condensate type occurs before subjecting a cellular composition to a stimulus. In some embodiments, the step of subjecting a cellular composition to a condition that promotes the formation of a condensate type occurs after subjecting a cellular composition to a stimulus, and before measuring a feature of at least one marking in the cellular composition.

Methods of forming condensates are known. For examples, cellular stress can cause the formation of stress granules. Examples of cellular stress include arsenate treatment, a temperature change, or a pH change. Accordingly, in some embodiments, causing the formation of the one or more target condensates comprises contacting the cellular composition with arsenate, an acid, or a base or altering the temperature of the cellular composition. In some embodiments, the stimulus includes a factor to promote the formation of a condensate type.

In some embodiments, the methods described herein do not include an independent step of forming a condensate. For example, in some embodiments, the method comprises subjecting a cellular composition comprising a cell type to the stimulus, and measuring a feature of at least one marker in at least a portion of the stimulus-subjected cellular composition, without further including another condensate formation step, e.g., application of a stress.

### B. Markers, Cell Staining, and Imaging Techniques

In certain aspects, the methods provided herein comprise measuring at least one condensate component, such as via a marker, in at least a portion of a stimulus-subjected cellular composition. In some embodiments, the feature of a condensate component is measured, such as via a marker. As described herein, the feature of a condensate component, such as via a marker, is assessed across two or more locations, including two or more pixels, that enables the determination of a change in location of a condensate component associated with subjecting a cellular composition to a stimulus. In some embodiments, the change in a feature of a condensate component, such as measured via a marker, is indicative of a change in a condensate characteristic. These and other features associated with markers and methods of measuring are discussed in more detail in the subsections below.

In some embodiments, the feature of a condensate component is based on a pixel analysis. In some embodiments, the pixel analysis is performed on a region of a cell, such as the nucleus and/or cytosol, or a region thereof. In some embodiments, the pixel analysis is performed without identification of individual condensates or features thereof (such as the boundary of a condensate). The pixels used for pixel analyses described herein can be any shape and size with respect to constraints imparted by the analyzed region. For example, in some embodiments, the region analyzed contains at least 2, such as at least any of 10, 100, 1,000, 5,000, 10,000, 20,000, 30,000, 40,000, or 50,000, pixels, such as within the boundary of the nucleus or cytosol of an image of a cell. In some embodiments, the pixels are uniformly sized. In some embodiments, the pixels are uniformly shaped.

### 1. Markers

As described herein, condensates comprise one or more components, such as macromolecules, e.g., a polypeptide and/or nucleic acid such as DNA or RNA. The methods provided herein comprise techniques for measuring, such as detecting, a condensate component, whether or not the condensate component is located in the condensate, and the term condensate component is not meant to imply that the component is within a condensate at the time of measurement. For example, in some embodiments, the cell type comprises a condensate type comprising polypeptide A, wherein additional polypeptide A is located within the cell type—for ease of describing the technology, the entire population of polypeptide A can be described as a condensate component to reflect the potential for polypeptide A located outside of the condensate type to be incorporated into the condensate type.

Markers are useful for determining the location and/or amount of a condensate and/or a condensate component, such as using an imaging technique. In some embodiments, the marker enables detection and/or measure (directly or indirectly) of a condensate component. For example, in some embodiments, the condensate component comprises a detectable feature that serves as a marker, which provides for direct detection and/or measure of said condensate component. In some embodiments, the detectable feature of the condensate component is inherent to the condensate component, e.g., a fluorophore that is a native feature of the condensate component. In some embodiments, the detectable feature of the condensate component is not natively present on or with the condensate component. For example, in some embodiments, the condensate component comprises a label, such as a fusion fluorescent label, e.g., GFP or mCherry. In some embodiments, the marker is an agent with affinity to a condensate component, e.g., an antibody, aptamer, or nucleic acid, which provides for indirect detection and/or measure of said condensate component. In some embodiments, the agent with affinity to a condensate component comprises a detectable label. In some embodiments, the marker is capable of detecting a condensate component associated with a condensate type (i.e., in the dense phase). In some embodiments, the marker is capable of detecting a condensate component not associated with a condensate type (i.e., in the light phase).

In some embodiments, the marker is specific for a condensate type. In some embodiments, the marker is specific for a subset of condensate types. For example, a condensate component may partition into a single condensate or a relatively small number of condensates, and detecting and/or measuring such a condensate component serves as a useful marker for assessing the selectivity of one or more stimuli on a condensate type or component thereof.

In some embodiments, the marker comprises a detectable label that can be detected and/or measured using a microscopy technique. In some embodiments, the label is a radioactive label, a colorimetric label, a luminescent label, a chemically-reactive label (such as a component moiety used in click chemistry), or a fluorescent label. In some embodiments, the marker comprises a condensate component fused with a label, such as fluorescent label. In some embodiments, the fusion condensate component comprises a Halo, Dendra2, GFP, RFP, or mCherry moiety. In some embodiments, the marker is an antibody comprising a detectable label, such as a primary labeled antibody. In some embodiments, the marker is an antibody detection system, including a system comprising a primary antibody that binds to a condensate component and a labeled secondary antibody that binds to the primary antibody.

In certain aspects, provided herein are methods for selecting and/or validating a marker, such as an antibody that specifically binds to a condensate component. In some embodiments, the method comprises obtaining one or more markers comprising an affinity agent, such as a primary labeled antibody and/or an antibody detection system, for one or more condensate components of a condensate type. In some embodiments, at least one of the one or more condensate components is a scaffold condensate component. Using the one or more markers, individually or in combination(s), a cellular composition comprising a cell type (with and/or without subjecting to a stimulus) is stained to assess the capability of each marker to label the associated condensate component whether in and/or out of the condensate type. Using the one or more markers, individual or in combination(s), a cellular composition comprising a modified version of the cell type having a knockdown (such as using siRNA) or knockout (such as using CRISPR) of associated condensate components (with and/or without subjecting to a stimulus) is stained to assess for any non-specific labeling of the marker. In some embodiments, the marker is found suitable for use in the methods described herein when the marker detects the associated condensate component in and out of the condensate and has a substantially low-level of non-specific labeling. In some embodiments, markers comprising an affinity agent, such as an antibody, are obtain such that there are more than one species of origin of the affinity agent, such as an antibody from a rabbit and an antibody from a mouse.

In some embodiments, when more than one condensate components is assessed it is helpful to utilize antibodies from different species, e.g., an mouse antibody specific to a first condensate component and a rabbit antibody specific to a second condensate. Such approaches enable co-staining. In some embodiments, different labeling techniques are used to enable co-localization measurements, e.g., a GFP fusion condensate component and an antibody marker. In some embodiments, sequential staining of a cellular composition is performed to enable co-localization measurements, e.g., staining using a first antibody marker, followed by stripping of the first antibody marker, followed by staining using a second antibody marker.

In some embodiments, the method comprises measuring a feature of a marker. In some embodiments, the method comprises measuring a feature of more than one marker. In some embodiments, measuring a feature of more than one marker comprises measuring a feature of a first marker and a second marker. In some embodiments, the first marker and the second marker label condensate components of a single condensate type. In some embodiments, the first marker and the second marker label condensate components of different condensate types.

### 2. Cell Staining and Imaging Techniques

Provided herein are methods for cell staining, such as to measure a marker, and methods for measuring a feature of at least one marker, such as using an imagine technique.

In some embodiments, the methods provided herein comprises subjecting a cellular composition or a derivative thereof, such as a fixed stimulus-subjected cellular composition, to one or more agents to detect one or more condensate components. For example, in some embodiments, the method comprises subjecting a fixed stimulus-subjected cellular composition to one more antibodies, wherein each of the one or more antibodies specifically binds to a condensate component (primary antibody). In some embodiments, the antibody comprises a detectable label, such as detectable via an imaging technique. In some embodiments, the method further comprises subjecting the fixed stimulus-subjected cellular composition to one or more secondary antibodies, wherein each secondary antibody comprises a detectable label. In some embodiments, the method involves using primary antibodies from different organisms, such as to enable co-staining techniques, e.g., to visualize co-localization of condensate components. In some embodiments, sequential staining of a cellular composition is performed to enable co-localization measurements, e.g., staining using a first antibody marker, followed by stripping of the first antibody marker, followed by staining using a second antibody marker. In some embodiments, the detectable label is a fluorescent label or a colorimetric label. In some embodiments, the staining technique is an immunofluorescent (IF) staining technique.

In some embodiments, the measuring a feature of the at least one marker in at least a portion of a stimulus-subjected cellular composition comprises imaging a stained stimulus-subjected cellular composition or an aspect thereof. In some embodiments, the imaging comprises a fluorescent imaging technique. In some embodiments, the imaging technique comprises any one or more of immunofluorescence (IF), in situ hybridization (ISH, such as FISH), gene fusion (e.g., GFP labeling), or a dye that is specific a condensate component. In some embodiments, the imaging technique comprises use of an agent to visualize a cellular feature, such as a cell membrane or an organelle.

In some embodiments, the imaging technique comprises use of an immunofluorescence (IF) technique, such as using an affinity label, such as a labeled antibody, that specifically binds to a condensate component. In some embodiments, the IF technique comprises subjecting a cellular composition or a derivative thereof to an affinity label, such as a labeled antibody, and imaging the cellular composition or the derivative thereof. In some embodiments, the imaging technique comprises use of an in situ hybridization (ISH) technique, e.g., fluorescent ISH (FISH) technique, such as using a nucleic acid probe that specifically binds to a condensate component. In some embodiments, the FISH technique comprises subjecting a cellular composition or a component thereof to a nucleic acid probe, and imaging the cellular composition or the derivative thereof. In some embodiments, the IF and/or FISH technique is performed in a high-throughput manner. In some embodiments, the methods further comprise use of an additional marker and/or dye to identify a feature of a cell model, such as a boundary of a cell bilayer and/or organelle.

In some embodiments, the imagining technique comprises use of a microscopy technique (and associated microscopy instrumentation). In some embodiments, the microscopy technique comprises a confocal microscopy technique. In some embodiments, the microscopy technique comprises a fluorescence microscopy technique. In some embodiments, the microscopy technique comprises a high-resolution microscopy technique. In some embodiments, the microscopy technique comprises a stimulated emission depletion (STED) microscopy technique. In some embodiments, the microscopy technique comprises a SoRa super-resolution spinning-disk microscopy technique. In some embodiments, the microscopy technique comprises an electron microscopy technique (such as cryo-EM or cryo-ET). In some embodiments, the microscopy technique comprises a total internal reflection fluorescence (TIRF) microscopy technique.

In some embodiments, the imaging is performed at one or more of the following magnifications 10×, 20×, 30×, 40×, 50×, 60×, 70×, 80×, 90×, or 100×.

In some embodiments, the imaging is performed such that a cellular composition is imaged using one or more fields of view. In some embodiments, the imaging is performed such that a cellular composition is imaged using one field of view.

In some embodiments, the imagine is performed such that a cellular composition (including a field of view thereof) is imaged at one or more Z-planes, including 1, 2, 3, 4, 5, 6, 7, 8, 9 or 10 Z-planes.

### C. Marker Features and Scoring Techniques

In certain aspects, the methods provided herein comprise measuring a feature of at least one marker in at least a portion of a stimulus-subjected cellular composition. In certain aspects, the measured feature is useful for various scoring techniques taught herein, such as a marker perturbation score and global condensate perturbation score. In some embodiments described herein, the feature comprises a distribution.

In some embodiments, the method comprises measuring a feature of a marker in an area of an image containing information regarding the location of the marker associated with a condensate and/or condensate component. In some embodiments, the measuring comprising extracting information of a feature of a marker in an area of an imagine. In some embodiments, the feature is assessed between two or more locations within an area of an image, including between two or more pixels. In some embodiments, the feature or features, such as a distribution, enable the determination of a change in location of a condensate component associated with subjecting a cellular composition to a stimulus. In some embodiments, the feature, such as a distribution, is assessed using a coefficient of variation (CV) calculation, such as determined via the standard deviation of a signal attributable to a marker in an area divided by the mean signal attributable to a marker in the area. In some embodiments, the area of the image assessed may be any area of a cellular composition or a derivative thereof, such as a fixed stimulus-subjected cellular composition. In some embodiments, the area is based on a cellular feature, such as a cell membrane or an organelle. For example, in some embodiments, the area is the nucleus of one or more cells of the cellular composition or derivative thereof In some embodiments, the area is arbitrarily defined, such as based on a field of view or a portion thereof.

In some embodiments, the method comprises measuring one or more features of two or more markers, such as any of 2, 3, 4, 5, 6, 7, 8, 9, 10, 11, 12, 13, 14, 1,5 16, 17, 18, 19, or 20 markers. Based on the teachings provided herein, one of ordinary skill in the art will readily appreciate that there is no theoretical limit to the number of markers that can be assessed, including the number of markers for which feature(s) are measured. In some embodiments, the measuring a feature step measures one or more features for two markers for each of the at least one or more condensate types in at least a portion of the stimulus-subjected cellular composition. In some embodiments, the measuring a feature step measures one or more features for at least one marker for at least two or more condensate types.

In some embodiments, the method comprises analyzing one or more obtained images from an imaging technique. In some embodiments, analysis techniques may comprise an automated or semi-automated analysis technique, such as for detecting a condensate and/or condensate component and/or cellular feature. In some embodiments, the analysis technique comprises an in silico analysis technique, such as for detecting a condensate and/or condensate component and/or cellular feature. Certain methods for in silico analysis, such as those incorporating machine learning and deep learning, are known in the art, such as US Pat. No. 10,303,979, which is hereby incorporated by reference herein in its entirety. In some embodiments, the image analysis comprises use of a software for determining one or more features of one or more markers, e.g., Harmony high-content imaging and analysis software (<https://www.perkinelmer.com/product/harmony-4-8-office-hh17000001>;accessed Aug. 1, 2023)

### 1. Marker Perturbation Scores

In some embodiments, exposing a cellular composition comprising a cell type to a stimulus may result in a change to a condensate, and such change can be measured using a known marker of said condensate (e.g., a condensate component or an agent that detects a condensate component). Accordingly, in some embodiments, the marker perturbation score is based on alterations (or a lack thereof) in the feature(s) of a marker for a condensate type following exposure to a stimulus, which may be indicative of the stimulus having an effect on the condensate or a component thereof. For example, after a cellular composition comprising a cell type is exposed to a stimulus, the feature(s) of a marker known to associate with a condensate type can be assessed for at least a portion of the cellular composition, such as a field of view (or a portion thereof). In some embodiments, the feature is based on a distribution, which is assessed using a coefficient of variation (CV) calculation, such as determined via the standard deviation of a signal attributable to a marker in an area divided by the mean signal attributable to a marker in the area.

As described herein, in some embodiments, more than one marker is assessed for a cellular composition or aliquots thereof. In some embodiments, the method further comprises determining a second marker perturbation score for a second marker in the stimulus-subjected cellular composition based on a measured feature(s) of the second marker. In some embodiments, the marker perturbation score is indicative of whether the stimulus modulates association of the marker with the at least one of the one or more condensate types in the cellular composition, and wherein the second marker perturbation score is indicative of whether the stimulus modulates association of the second marker with the at least one of the one or more condensate types in the cellular composition. In some embodiments, the marker and the second marker associate with the same condensate type. In some embodiments, the marker and the second marker associate with the different condensate types.

In some embodiments, each marker perturbation score is independently based on a coefficient of variation (CV) for the marker, and wherein the CV is determined based on a standard deviation (STD) of distribution intensity for the marker divided by the mean distribution intensity of the marker. In some embodiments, the distribution intensity for each marker is based on a pixel analysis of one or more images of the one or more cellular compositions, or a portion of each thereof.

In some embodiments, the method further comprises evaluating a reference stimulus, wherein the method comprises determining a reference marker perturbation score. In some embodiments, the reference marker perturbation score is 0, and wherein the marker perturbation score of >0 is indicative of the stimulus having a condensate modulatory characteristic.

In some embodiments, the marker perturbation score is obtained by normalizing and/or bucketing one or more underlying distribution measurements, such as based on a CV calculation. In some embodiments, the marker perturbation score is based on a median absolute deviation (MAD) Z-score of the CV for the marker. In some embodiments, the method further comprises obtaining the MAD Z-score for the marker. In some embodiments, the marker perturbation score is 1 when the MAD Z-score is >5 or <−5. In some embodiments, the marker perturbation score is 0.5 when 2.5 <MAD Z-score≤5 or −5≤MAD Z-score <−2.5. In some embodiments, the marker perturbation score is 0 when −2.5≤MAD Z-score≤2.5.

In some embodiments, the marker perturbation score is based on calculating a modified Z-score, e.g., MAD Z-score, for each of one or more features of a marker, e.g., wherein the one or more features are each selected from a texture feature, an intensity feature, and/or a morphology feature. In some embodiments, the modified Z-score is a Median Absolute Deviation (MAD) Z-score. In some embodiments, the modified Z-score is (Xi-\tilde{X})/MAD, where Xi was a single data value, \tilde{X} was the median of the negative control treated wells, and MAD was Median Absolute Deviation of the negative control treated wells. In some embodiments, the method comprises extracting information of a feature, such as by using software for determining one or more features of one or more markers, e.g., Harmony high-content imaging and analysis software. In some embodiments, the method comprises extracting information one or more features from a set of images (or data therefrom). In some embodiments, the one or more features from the set of images (or data therefrom) is the same for images originating from differently treated cellular compositions. In some embodiments, the one or more features are based on one or more of a texture feature, an intensity feature or a morphology feature. Such features are generally known in the art and used (and described) in, e.g., the Harmony high-content imaging and analysis software instruction manual, which is hereby incorporated herein by reference in its entirety. Additional description of content screening useful for feature analysis described herein can be found in Letzsch et al., “Phenotypic Profiling of Autophagy Using Opera Phenix High-Content Screening System,” Application Note, 2016, PerkinElmer, which is hereby incorporated herein by reference in its entirety. For example, in brief, texture features measure the degree and nature of textures within images and objects to quantify their roughness and smoothness. Any type of texture measures intensity variations in an image. An object or entire image without much texture has a smooth appearance, and an object or image with a lot of texture will appear rough and show a wide variety of pixel intensities. In the context of condensates, if a recorded measurement for a given texture feature has a high value one can infer roughness and changes in the intensity in that object or image, and thus in a compound treatment if a texture measurement increases it can infer more puncta or that a larger puncta was broken down to several smaller puncta. In some embodiments, the texture feature is a Gabor maximum, Gabor minimum, Haralick contrast, Haralick correlation, Haralick homogeneity, Haralick sum variance, SER bright, SER dark, SER edge, SER hole, SER ridge, SER saddle, SER spot, or SER valley). In some embodiments, the feature is an intensity feature, such as measured via one or more of a coefficient of variation, mean intensity, contrast, standard deviation. In some embodiments, the feature is a morphology feature, such as based on one or more of an area, roundness, perimeter, width, length, or width to length ratio.

In some embodiments, the modified Z-score, e.g., MAD Z-score, is independently calculated for each assessed feature of each marker. Subsequently, the method may comprise determining a feature-change score for each feature of each marker based on the associated modified Z-score, e.g., MAD Z-score, and a proportional scale. As described herein, the proportional scale can be arbitrarily determined and one of ordinary skill in the art will readily appreciate the scope of such scale. For example, in some embodiments, the proportional scale is −5 to 5. In some embodiments, the proportional scale is 0 to 1. Following the determination of a feature-change score, the method described herein may then comprise aggregating feature-change scores of a top percentage of the feature-change scores to determine the marker perturbation score. In some embodiments, the aggregation is performed by summing. In some embodiments, the aggregation is performed by averaging. In some embodiments, the set of feature-change scores used to generate a marker perturbation score is based on a threshold or by taking the top percentage of feature-change scores, e.g., the top of any of 50%, 45%, 40%, 35%, 30%, 25%, 20%, 15%, 10%, or 5%. In some embodiments, all feature-change scores are used to generate a marker perturbation score. In some embodiments, the marker perturbation score reflects how strongly a treatment condition perturbed a single condensate marker as compared to a reference

In certain aspects, provided is a method of identifying a stimulus for a condensate modulatory characteristic for one or more condensate types, the method comprising: (a) subjecting a cellular composition comprising a cell type to the stimulus, (b) measuring a feature (including one of more) of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured feature of each marker, wherein the determining comprises: (i) determining a modified Z-score (such as a MAD Z-score) for the feature (or each feature) of each marker; (ii) determining a feature-change score for the feature (or each feature) for each marker based on the associated modified Z-score, e.g., MAD Z-score, and a proportional scale; and (iii) aggregating (such as by sum or average) the feature-change scores for each marker to determine the marker perturbation score; and (d) identifying the stimulus for the condensate modulatory characteristic from the marker perturbation score. In certain aspects, provided herein is a method for determining a marker perturbations score, the method comprising (a) measuring a feature (including one of more) of at least one marker in at least a portion of a stimulus-subjected cellular composition, wherein the marker associates with at least one of the one or more condensate types prior to and/or after the cellular composition is subjected to the stimulus; and (b) determining a marker perturbation score for each marker in the stimulus-subjected cellular composition based on the measured feature of each marker, wherein the determining comprises: (i) determining a modified Z-score (such as a MAD Z-score) for the feature (or each feature) of each marker; (ii) determining a feature-change score for the feature (or each feature) for each marker based on the associated modified Z-score, e.g., MAD Z-score, and a proportional scale; and (iii) aggregating (such as by sum or average) the feature-change scores for each marker to determine the marker perturbation score. In some embodiments, the method further comprises determining a global condensate perturbation score, such as using a method taught herein. In some embodiments, the measuring includes extracting information of the feature, such as by using a software for assess features including any one or combination of one or more texture features, one or more intensity features, or one or more morphology features. In some embodiments, the one or more texture features include one or more of Gabor maximum, Gabor minimum, Haralick contrast, Haralick correlation, Haralick homogeneity, Haralick sum variance, SER bright, SER dark, SER edge, SER hole, SER ridge, SER saddle, SER spot, or SER valley). In some embodiments, the one or more intensity features include one or more of coefficient of variation, mean intensity, contrast, standard deviation. In some embodiments, the one or more morphology features include one or more of area, roundness, perimeter, width, length, or width to length ratio.

In some embodiments, each marker perturbation score is independently based on at least one characteristic associated with at least one of the one or more condensate types and/or the markers.

### 2. Global Condensate Perturbation Scores

In some embodiments, the method involves a global condensate score. In some embodiments, the global condensate score is a metric useful for assessing the selectivity of a stimulus across one or more condensate types, such as any of 2, 3, 4, 5, 6, 7, 8, 9, 10, 11, 12, 13, 14, 15, 16, 17, 18, 19, or 20 condensate types. Based on the teachings provided herein, one of ordinary skill in the art will readily appreciate that there is no theoretical limit to the number of marker perturbation scores and/or stimuli that can be assessed using a global condensate perturbation score.

In some embodiments, the global condensate perturbation score is indicative of the stimulus selectively modulating the one or more condensate types for the stimulus-subjected cellular composition. In some embodiments, the global condensate perturbation score is indicative of the stimulus non-selectively modulating the one or more condensate types for the stimulus-treated cellular composition. In some embodiments, the global condensate perturbation score is indicative of the stimulus not substantially modulating the one or more target condensate types for the stimulus-treated cellular composition. In some embodiments, the global condensate perturbation score is based on two or more marker perturbation scores, such as a first marker perturbation score and a second marker perturbation score.

In some embodiments, the global condensate perturbation score is calculated by dividing [a sum of all marker perturbation scores for all markers] with [the number of all markers] (i.e., an average) In some embodiments, the global condensate perturbation score is calculated by dividing [a sum of all marker perturbation scores determined for all markers of two or more different target condensates] with [the number of all markers of the two or more different target condensates]. In some embodiments, the global condensate perturbation score is calculated by summing marker perturbation scores for the markers (e.g., the markers of interest, such as those meeting a certain threshold or a top % of scores).

In some embodiments, the method comprises determining a reference global condensate perturbation score. In some embodiments, the global condensate perturbation score is based on a Z-score, such as a median-based Z-score. In some embodiments, the reference global condensate perturbation score is 1, and wherein: (i) the global condensate perturbation score of 0< and <1 is indicative of the stimulus selectively modulating the one or more condensate types in the cellular composition; and (ii) the global condensate perturbation score of 0 is indicative of the stimulus not substantially modulating the one or more condensate types in the cellular composition.

In some embodiments, when the global condensate perturbation score of 0< and <1, the smaller the global condensate perturbation score for the stimulus the higher the selectivity of the stimulus.

### 3. Comparisons

In certain aspects, the methods provided herein comprise comparisons between condensate components and/or condensate types, such as enabled by marker perturbation scores and global condensate perturbation score.

In some embodiments, the method comprises subjecting a cellular composition to a first stimulus and a second stimulus (such as via two wells individual containing aliquots of the cellular composition), measuring a feature of at least one marker in at least a portion of a first and second stimulus-subjected cellular composition, and independently determining a marker perturbation score for each marker in the first and second stimulus-subjected cellular composition based on the measured feature of each marker in the first and second stimulus-subjected cellular composition. In some embodiments, the method comprises comparing a marker perturbation score for a marker in a first stimulus-subjected cellular composition with a marker perturbation score for a marker in the second stimulus-subjected cellular composition. In some embodiments, the method comprises comparing global condensate perturbation score for a cellular composition subjected across two or more stimuli. In some embodiments, the methods comprises determining two or more second global condensate perturbation scores, such as to evaluate two or more stimuli.

### III. KITS AND COMPOSITIONS

In certain aspects, provided herein are kits and composition useful for the methods described herein. In some embodiments, provided herein is a kit comprising one or more agents, such as an antibody, that recognize one or more condensate components, e.g., a marker panel. In some embodiments, the marker panel comprises two or more antibodies, wherein two or more antibodies recognize the same condensate component. In some embodiments, the marker panel comprises two or more antibodies, wherein two or more antibodies recognize different condensate components. In some embodiments, the kit comprises one or more cellular composition useful for the methods described herein.

### IV. SYSTEMS

In certain aspects, provided herein are systems for performing aspects of the methods taught herein.

In some embodiments, the system comprises: one or more processors; and memory storing one or more programs, the one or more programs configured to be executed by the one or more processors, the one or more programs including instructions for: receiving one or more image data packages comprising one or more images of a stimulus-subject cellular composition; applying one or more processes to the received image data package(s) to measure one or more features of one or more markers; and storing and/or outputting the one or more features in one or more feature data packages. In some embodiments, at least one image of a stimulus-subject cellular composition is stained using an agent, such as an antibody recognizing a condensate component. In some embodiments, the system is configured to automate at least a portion of the instructions therein, such as a identifying areas of a cellular composition or derivative thereof. In some embodiments, the system comprises instructions for: determining a marker perturbation score for each marker in the stimulus-subjected cellular composition based on the measured feature of each marker stored in one or more feature data packages; and storing and/or outputting the marker perturbation score in a marker perturbation data package. In some embodiments, the system comprises instructions for: determining a global condensate perturbation score, wherein the global condensate perturbation score is based on at least one marker perturbation score; and storing and/or outputting the global condensate perturbation score in a global condensate perturbation data package. In some embodiments, the system comprises instruction for: selecting a stimulus exhibiting selectivity (relative to condensate type and/or condensate component), wherein the selecting the stimulus exhibiting selectivity is based on one or more marker perturbation scores and/or one or more global condensate perturbation scores. In some embodiments, the system comprises a user-interface, such as a screen, touchpad, and/or buttons or keys for providing commands and/or reviewing information related to the computer-implementation of the methods provided herein.

### V. FURTHER ASPECTS ENABLED BY THE DISCLOSURE HEREIN

In other aspects, provided herein are further methods and embodiments enabled by the disclosure herein.

In some embodiments, provided herein is a method of identifying a compound (such as a small molecule therapeutic candidate) that modulates a condensate type, including selective modulates, e.g., modulates a first condensate type without modulating a second condensate type, the method comprising identifying a stimulus for a condensate modulatory characteristic for one or more condensate types as described herein.

In some embodiments, provided herein is a method of identifying a compound useful for treating a disease, the method comprising identifying a stimulus for a condensate modulatory characteristic for one or more condensate types as described herein, wherein the stimulus causing the condensate modulatory characteristic is useful for treating the disease.

In some embodiments, provided herein is a method of identifying a condensate type associated with a disease, the method comprising identifying a stimulus for a condensate modulatory characteristic for one or more condensate types as described herein, wherein the stimulus is a factor associated with the disease.

In some embodiments, provided herein is a method of identifying one or more interactions of a compound, or a portion thereof, and a condensate type, or a component thereof, the method comprising identifying a stimulus for a condensate modulatory characteristic for one or more condensate types as described herein.

In some embodiments, provided herein is a method of identifying a molecular target for a therapeutic drug useful for treating a disease, the method comprising identifying a condensate of interest using any one of the methods described herein; and identifying the molecular target based on an association and/or interaction with the condensate of interest.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### VI. EXEMPLARY EMBODIMENTS

### Embodiment 1

A method of identifying a stimulus for a condensate modulatory characteristic for one or more condensate types, the method comprising. (a) subjecting a cellular composition comprising a cell type to the stimulus, (b) measuring a distribution of at least one marker in at least a portion of the stimulus-subjected cellular composition, wherein the marker associates with at least one of the one or more condensate types prior to and/or after the cellular composition is subjected to the stimulus; (c) determining a marker perturbation score for each marker in the stimulus-subjected cellular composition based on the measured distribution of each marker; and (d) identifying the stimulus for the condensate modulatory characteristic from the marker perturbation score.

### Embodiment 2

The method of embodiment 1, further comprising the step of: determining a global condensate perturbation score, wherein the global condensate perturbation score is based on at least one marker perturbation score; and the identifying the stimulus for the condensate modulatory characteristic step can be identified from at least one marker perturbation score and/or global condensate perturbation score.

### Embodiment 3

The method of any one of the preceding embodiments, wherein the measuring distribution step measures two markers for each of the at least one or more condensate types in at least a portion of the stimulus-subjected cellular composition.

### Embodiment 4

The method of any one of the preceding embodiments, wherein the measuring distribution step measures at least one marker for at least two or more condensate types.

### Embodiment 5

The method of any one of embodiments 1-4, wherein each marker is independently a lipid, a polypeptide, or a nucleic acid.

### Embodiment 6

The method of any one of embodiments 1-4, wherein each marker is independently a polypeptide.

### Embodiment 7

The method of any one of the preceding embodiments, wherein each marker: (i) is within the condensate type; (ii) partitions into the condensate type after subjecting the cellular composition to the stimulus; or (iii) is excluded from the condensate type after subjecting the cellular composition to the stimulus.

### Embodiment 8

The method of any one of embodiments 1-7, wherein each marker is independently a condensate scaffold polypeptide or nucleic acid.

### Embodiment 9

The method of any one of embodiment 8, wherein at least one marker is a condensate scaffold polypeptide.

### Embodiment 10

The method of any one of embodiments 1-7, wherein each marker is independently a condensate client polypeptide or nucleic acid.

### Embodiment 11

The method of any one of the preceding embodiments, wherein the measuring a distribution of the at least one marker in at least a portion of the stimulus-subjected cellular composition comprises staining at least a portion of the cellular composition for the marker.

### Embodiment 12

The method of embodiment 11, wherein the staining is immunofluorescent (IF) staining.

### Embodiment 13

The method of any one of the preceding embodiments, wherein the measuring, for at least the portion of the cellular composition, the distribution of the at least one marker comprises imaging at least the portion of the cellular composition.

### Embodiment 14

The method of embodiment 13, wherein the imaging comprises a fluorescent imaging technique.

### Embodiment 15

The method of any one of the preceding embodiments, wherein at least one of one or more different condensate types form in the cellular compositions after being subjected the stimulus.

### Embodiment 16

The method of any one of the preceding embodiments, wherein the stimulus is selected from the group consisting of an exogenous compound, exogenous peptidic material, exogenous genetic material, stressors, an environmental stimulus, and combinations thereof.

### Embodiment 17

The method of any one of the preceding embodiments, wherein the stimulus is an exogenous compound.

### Embodiment 18

The method of embodiment 17, wherein the compound is a small molecule therapeutic candidate, or precursor thereof.

### Embodiment 19

The method of embodiment 17 or 18, wherein the cellular composition is subjected to the compound at a known concentration.

### Embodiment 20

The method of any one of the preceding embodiments, further comprising subjecting the cellular composition to a second stimulus, measuring a distribution of the at least one marker in at least a portion of the second stimulus-subjected cellular composition, and independently determining a marker perturbation score for each marker in the second stimulus-subjected cellular composition based on the measured distribution of each marker in the second stimulus-subjected cellular composition.

### Embodiment 21

The method of embodiment 20, further comprising comparing a marker perturbation score for a marker in the stimulus-subjected cellular composition with a marker perturbation score for a marker in the second stimulus-subjected cellular composition.

### Embodiment 22

The method of embodiment 20 or 21, further comprising determining a second global condensate perturbation score to evaluate the second stimulus for the condensate modulatory characteristic, wherein the second global condensate perturbation score is based on at least one marker perturbation score for a marker in the second stimulus-subjected cellular composition.

### Embodiment 23

The method of embodiment 22, further comprising comparing the global condensate perturbation score with the second global condensate perturbation score.

### Embodiment 24

The method of any one of the preceding embodiments, further comprising determining at least one characteristic associated with at least one of the one or more condensate types and/or the markers.

### Embodiment 25

The method of embodiment 24, wherein the at least one characteristic associated with the at least one of the one or more condensate types and/or the markers comprises: (i) location of the condensate type; (ii) distribution of the condensate type and/or the marker; (iii) number of the condensate type; (iv) size of the condensate type; (v) ratio of the amount of the condensate type and a control condensate; (vi) a functional activity associated with the condensate type; (vii) composition of the condensate type, (viii) co-localization of the condensate type with a biomolecule; (ix) diffusion coefficient of a component of the condensate type; (x) stability of the condensate type; (xi) dissolution or reduction in size of the condensate type; (xii) surface area of the condensate type; (xiii) sphericity of the condensate type; (xiv) liquidity of the condensate type; (xv) solidification of the condensate type; (xvi) location of the marker; (xvii) amount of the marker or a precursor thereof; (xviii) condensate partitioning of the marker into the condensate type: (xix) a functional activity associated with the marker; (xx) aggregation of the marker; (xxi) post-translational modification status of the marker; and (xxii) amount of a degradation product of the marker.

### Embodiment 26

The method of any one of embodiments the preceding embodiments, wherein at least one of the one or more condensate types is selected from the group consisting of a cleavage body, p-granule, histone locus body, a multivesicular body, neuronal RNA granule, nuclear gem, nuclear pore complex, nuclear speckle, nuclear stress body, nucleolus, Oct1/PTF/transcription (OPT) domain, paraspeckle, perinucleolar compartment, PML nuclear body, PML oncogenic domain, polycomb body, processing body. Sam68 nuclear body, stress granule, splicing speckle, P62 body, Cajal body, heterochromatin, transcriptional condensate, and centrosome.

### Embodiment 27

The method of any one of the preceding embodiments, wherein the method comprises measuring a distribution of a second marker in at least a portion of the stimulus-subjected cellular composition, wherein the second marker associates with at least one of the one or more condensate types prior to and/or after the cellular composition is subjected to the stimulus.

### Embodiment 28

The method of embodiment 27, further comprising determining a second marker perturbation score for the second marker in the stimulus-subjected cellular composition based on the measured distribution of the second marker.

### Embodiment 29

The method of embodiment 27 or 28, wherein the marker perturbation score is indicative of whether the stimulus modulates association of the marker with the at least one of the one or more condensate types in the cellular composition, and wherein the second marker perturbation score is indicative of whether the stimulus modulates association of the second marker with the at least one of the one or more condensate types in the cellular composition.

### Embodiment 30

The method of any one of embodiments 27-29, wherein the marker and the second marker associate with the same condensate type.

### Embodiment 31

The method of any one of embodiments 27-29, wherein the marker and the second marker associate with the different condensate types.

### Embodiment 32

The method of any one of the preceding embodiments, wherein each marker perturbation score is independently based on at least one characteristic associated with at least one of the one or more condensate types and/or the markers.

### Embodiment 33

The method of any one of preceding embodiments, wherein each marker perturbation score is independently based on a coefficient of variation (CV) for the marker, and wherein the CV is determined based on a standard deviation (STD) of distribution intensity for the marker divided by the mean distribution intensity of the marker.

### Embodiment 34

The method of embodiment 33, wherein the distribution intensity for each marker is based on a pixel analysis of one or more images of the one or more cellular compositions, or a portion of each thereof.

### Embodiment 35

The method of embodiment 33 or 34, wherein each marker perturbation score is based on a median absolute deviation (MAD) Z-score of the CV for the marker.

### Embodiment 36

The method of embodiment 35, further comprising obtaining the MAD Z-score for the marker.

### Embodiment 37

The method of embodiment 35 or 36, wherein: (i) the marker perturbation score is 1 when the MAD Z-score is >5 or <−5; (ii) the marker perturbation score is 0.5 when 2.5<MAD Z-score≤5 or −5≤MAD Z-score<−2.5; and (iii) the marker perturbation score is 0 when −2.5≤MAD Z-score≤2.5.

### Embodiment 38

The method of any one of the preceding embodiments, further comprising evaluating a reference stimulus, wherein the method comprises determining a reference marker perturbation score.

### Embodiment 39

The method of embodiment 38, wherein the reference marker perturbation score is 0, and wherein the marker perturbation score of >0 is indicative of the stimulus having a condensate modulatory characteristic.

### Embodiment 40

The method of any one of embodiments 2-39, wherein the global condensate perturbation score is indicative of the stimulus selectively modulating the one or more condensate types for the stimulus-subjected cellular composition.

### Embodiment 41

The method of any one of embodiments 2-39, wherein the global condensate perturbation score is indicative of the stimulus non-selectively modulating the one or more condensate types for the stimulus-treated cellular composition.

### Embodiment 42

The method of any one of embodiments 2-39, wherein the global condensate perturbation score is indicative of the stimulus not substantially modulating the one or more target condensate types for the stimulus-treated cellular composition.

### Embodiment 43

The method of any one of embodiments 27-42, wherein the global condensate perturbation score is based on the marker perturbation score and the second marker perturbation score.

### Embodiment 44

The method of any one of embodiments 2-43, wherein the global condensate perturbation score is calculated by dividing [a sum of all marker perturbation scores] with [the number of marker perturbation scores].

### Embodiment 45

The method of any one of embodiments 2-44, wherein the method comprises determining a reference global condensate perturbation score.

### Embodiment 46

The method of embodiment 45, wherein the reference global condensate perturbation score is 1, and wherein: (i) the global condensate perturbation score of 0<and <1 is indicative of the stimulus selectively modulating the one or more condensate types in the cellular composition; and (ii) the global condensate perturbation score of 0 is indicative of the stimulus not substantially modulating the one or more condensate types in the cellular composition

### Embodiment 47

The method of embodiment 46, wherein for the global condensate perturbation score of 0< and <1, the smaller the global condensate perturbation score for the stimulus the higher the selectivity of the stimulus.

### EXAMPLES

### Example 1

This example demonstrates methodology for identifying a stimulus for a condensate modulatory characteristic for a plurality of condensate types. Specifically, stimuli having a selective condensate modulatory characteristic are identified using the methodology described herein.

HeLa cells were grown in a T175 flask at 37° C. and 5% CO₂ to 100% confluency in DMEM+GlutaMAX containing 10% FBS and 1% PS. 5 mL of TrypLe was used to remove the HeLa cells from the T175 flask after excess cell media was washed off with 5 mL of PBS. A 100,000 cell per mL solution was made to use for cell plating. Using a MultiFlo with a 5 μL cassette, 6 μL of media from the 100,000 cell/mL solution was plated into each well of a 1536-well plate. This resulted in 600 cells/well. The plate was then centrifuged at 1000 rpm for 1 minutes to ensure the media and cells reached the bottom of the wells. The plate was then stored in a humidified chamber within the cell culture incubator.

About 48 hours after plating, candidate compounds were dispensed onto the cells using a Beckman Echo 650/550 acoustic liquid handler. Cells were incubated in the humidified chamber in a cell culture incubator at 37° C. and 5% CO₂ for 6 hours.

Following the treatment period, the cells were fixed in the wells of the plate. Using a MultiFlo with a 1 μL cassette, 3 μL of 12% PFA was added directly to the cell media in the 1536-well plate for 10 minutes to fix the cells. The EL-406 liquid handler was used to aspirate fixation media and wash the plates with PBS. Three washes with PBS were completed. Following fixation, the cells were permeabilized. Using a EL-406 with a 5 μL cassette, the media in the 1536-well plate was aspirated and 6 μL of a PBS+0.5% BSA solution containing 0.5% Triton X-100 was added to the cells for 10 minutes. The EL-406 liquid handler was used to aspirate the media and wash the plates with PBS with 0.5% BSA. Three washes with PBS were completed.

Following fixation and permeabilization, the cells were stained using a two-antibody system (unlabeled primary antibody and labeled secondary antibody) and dyes for visualizing cellular features. Specifically, the primary antibodies were diluted in PBS+0.5% BSA to a working concentration and 6 μL of this solution was added to each well of the 1536-well plate using a MultiFlo with a 1 μL cassette with metal tips. Incubation with the primary antibodies was performed overnight at 4° C. Primary antibodies were then washed off using the EL-406, and the cells were washed three times with PBS containing 0.5% BSA. Next, secondary antibodies were diluted 1:500 in PBS+0.5% BSA and 6 μL of the solution was added to the each well of the plate using the MultiFlo with a 5 μL cassette. Secondary antibodies were incubated at room temperature for 1 hour. Secondary antibodies were then washed off using the EL-406 and the cells were washed three times with PBS+0.5% BSA. Following antibody staining, the cells were stained with Hoechst (nuclear stain) and CMB (cell mask blue). Specifically, 3 μL of a PBS+0.5% BSA containing 6 μg/mL Hoechst and 12 μg/mL CMB was added onto the 6 μL of PBS+0.5% BSA in the 1536-well plate using the Multiflo with a 3 μL cassette. The Hoechst and CMB solution was not washed off of the plate. Plates were sealed and stored in 4° C. until imaging.

Images of the stained cells were acquired on a Phenix Plus confocal microscope.

Using the obtained images, marker perturbation scores and global condensate perturbation scores were calculated using the coefficient of variation (CV) and Index of dispersion (D). Specifically, CV and D were calculated for both the nuclear and cytoplasmic cell compartments separately. CV was calculated as the STD/mean of pixel intensities in a given cell compartment. D was calculated as the STD\hat{ }2/mean of pixel intensities in a given cell compartment.

Next, a modified Z-score for each marker stained in each treatment condition tested was calculated using CV and D. The result was Z-scores for each treatment condition that describe how much CV or D differ compared to negative control treated wells. Z-scores were calculated using (Xᵢ-\tilde{X})/MAD, where Xᵢ was a single data value, \tilde{X} was the median of the negative control treated wells, and MAD was Median Absolute Deviation of the negative control treated wells. Marker perturbation scores were then calculated for each treatment condition for each marker stained. This score reflected how strongly a treatment condition perturbs a single condensate marker. Specifically, marker perturbation scores can be 0, 0.5, or 1, and are determined using Modified Z-score cutoffs. Modified Z-scores with an absolute value <2.5 received a marker perturbation score of 0. Modified Z-scores with an absolute value >2.5 but <5 received a marker perturbation score of 0.5. Modified Z-scores with an absolute value >5 received a marker perturbation score of 1.

Using the marker perturbation scores, a global condensate perturbation score was calculated for each treatment condition. This score reflected how broadly a treatment condition perturbs all condensates. The global condensate perturbation score range from 0 to 1, and were calculated as the sum of all marker perturbation scores divided by the total number of markers stained.

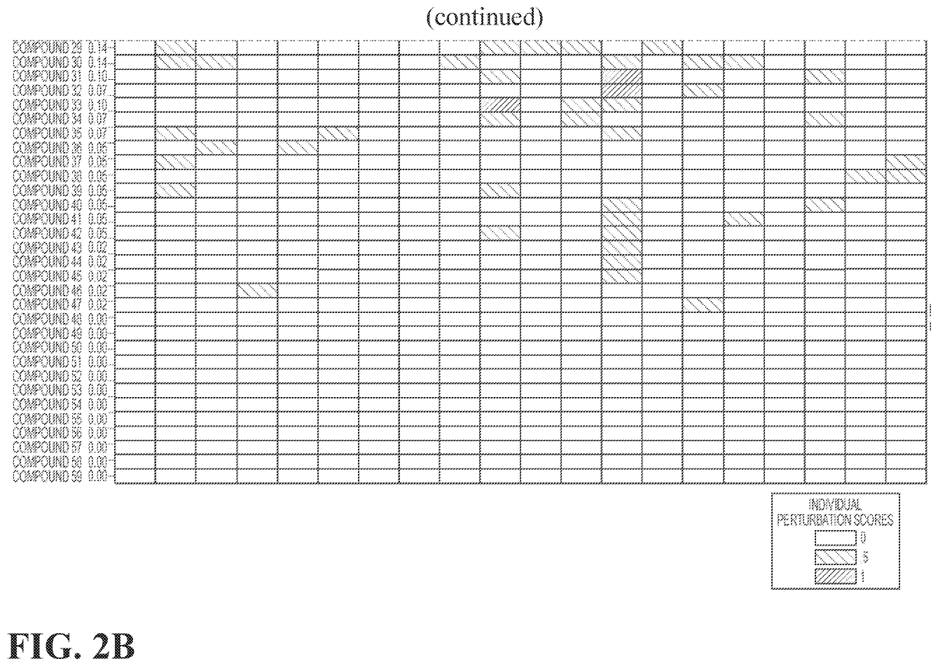

Marker perturbation scores and global condensate perturbation scores were visualized using Spotfire. As shown in FIG. 2, marker perturbation scores and global condensate perturbation scores for 60 candidate compounds at 4 μM concentration are assessed across 12 condensate types using two markers for each condensate type. As shown in FIG. 2, the different compounds tested have very different condensate specificity as demonstrated by the wide range of global condensate perturbation scores, and the methods provided herein enable, e.g., identification of selective condensate modulators.

### Example 2

This example demonstrates methodology for identifying a stimulus for a condensate modulatory characteristic for a plurality of condensate types. Specifically, stimuli having a selective condensate modulatory characteristic are identified using the methodology described herein.

### Experimental Methods

U2OS cells were grown in a T175 flask at 37° C. and 5% CO₂ to 90% confluency in DMEM containing 10% FBS and 1% PS. 5 mL of TrypLe was used to remove the U2OS cells from the T175 flask after excess cell media was washed off with 5 mL of PBS. A 62,500 cell per mL solution was made to use for cell plating. Using a Multidrop Combi with a small tube metal tip dispensing cassette, 8 μL of media from the 62,500 cell/mL solution was plated into each well of a 1536-well plate. This resulted in 500 cells/well. Plates were left at room temperature for 15 minutes to allow the cells to settle to the bottom of the plate before being placed in a humidified chamber within the cell culture incubator.

About 48 hours after plating, candidate compounds were dispensed onto the cells using a Beckman Echo 650 acoustic liquid handler. Cells were incubated in the humidified chamber in a cell culture incubator at 37° C. and 5% CO₂ for 6 hours.

Following the treatment period, using an EL-406, the media was aspirated, then a 4% PFA solution was dispensed using the syringe dispenser of the EL-406. The EL-406 was used to aspirate fixation media after 10 minutes and wash the cells with PBS three times. Following fixation, the cells were permeabilized. Using the EL-406, the PBS in the 1536-well plate was aspirated and 8 μL of a PBS+0.5% BSA solution containing 0.5% Triton X-100 was added to the cells for 10 minutes. The EL-406 was used to aspirate the media and wash the plates with PBS with 0.5% BSA three times

Following fixation and permeabilization, the cells were stained using a two-antibody system (unlabeled primary antibody and labeled secondary antibody) and dyes for visualizing cellular features. The two-antibody system used a first antibody specific to a first marker for a condensate type and a second antibody specific to a second marker for the same condensate type, wherein the first antibody and second antibody are from different organisms, e.g., mouse and rabbit. Specifically, the primary antibodies were diluted in PBS+0.5% BSA to a working concentration and 6 μL of this solution was added to each well of the 1536-well plate using a Multidrop Combi with a small tube metal tip dispensing cassette. Incubation with the primary antibodies was performed overnight at 4° C. Primary antibodies were then washed off using the EL-406, and the cells were washed three times with PBS containing 0.5% BSA. Next, secondary antibodies were diluted 1:1000 in PBS+0.5% BSA and 6 μL of the solution was added to the each well of the plate using a Multidrop Combi with a small tube metal tip dispensing cassette. Secondary antibodies were incubated at room temperature for at least 1 hour. Secondary antibodies were then washed off using the EL-406 and the cells were washed three times with PBS+0.5% BSA. Following antibody staining, the cells were stained with Hoechst (nuclear stain) and CMO (cell mask orange). Specifically, 6 μL of a PBS+0.5% BSA containing 6 μg/mL Hoechst and 12 μg/mL CMO was added onto cells after aspiration using the EL-406. The Hoechst and CMB solution was not washed off of the plate. Plates were sealed and stored in 4° C. until imaging.

Images of the stained cells were acquired on a Phenix Plus confocal microscope.

### Image Analysis Protocol Wherein the Global Condensate Perturbation Score is Based on Averages of Marker Perturbation Scores

Using the obtained images, marker perturbation scores and global condensate perturbation scores were calculated using multiple image features extracted by Harmony image analysis software. The conceptual goal of the marker perturbation score was to represent in a single number the degree to which a stimulus changes the staining pattern of a marker relative to a control by combining all the relevant information captured in the multiple extracted features. The conceptual goal of the global condensate perturbation score was to represent in a single number the degree to which a stimulus changes the staining pattern of all measured condensates relative to a control by combining all the individual marker perturbation scores. One quantitative method to achieve these goals is described below. All features were calculated in the two channels containing condensate marker staining for both the nuclear and cytoplasmic cell compartments separately. The features can be subdivided into 3 categories including texture (Gabor maximum, Gabor minimum, Haralick contrast, Haralick correlation, Haralick homogeneity, Haralick sum variance, SER bright, SER dark, SER edge, SER hole, SER ridge, SER saddle, SER spot, SER valley), intensity (coefficient of variation, mean intensity, contrast, standard deviation), and morphology (area, roundness, perimeter, width, length, width to length ratio).

Next, a modified Z-score for each feature assessed for each marker stained in each treatment condition tested was calculated. The result was an individual Z-score for each feature for each marker in each treatment condition that describes how much the feature differs compared to negative control treated wells. Z-scores were calculated using (Xi-\tilde{X})/MAD, where Xi was a single data value, \tilde{X} was the median of the negative control treated wells, and MAD was Median Absolute Deviation of the negative control treated wells. Marker perturbation scores were then calculated for each treatment condition for each marker stained. This score reflected how strongly a treatment condition perturbed a single condensate marker. Marker perturbation scores can range from 0 to 1 and are determined by aggregating the features that changed most relative to negative control. First, each feature was given an individual feature-change score. Features with modified Z-scores with an absolute value <3 received a feature-change score of 0. Features with modified Z-scores with an absolute value >10 received a feature-change score of 1. Features with modified Z-scores with an absolute value >3 but <10 received a feature-change score proportionally scaled between 0 and 1. The marker perturbation score was then calculated as the average of the top 25% feature-change scores. For example, if 20 features are measured, the marker perturbation score was calculated as the average of the 5 highest feature-change scores.

Using the marker perturbation scores, a global condensate perturbation score was calculated for each treatment condition. This score reflected how broadly a treatment condition perturbs all condensates. The global condensate perturbation score also ranges from 0 to 1, and was calculated as the average of all marker perturbation scores.

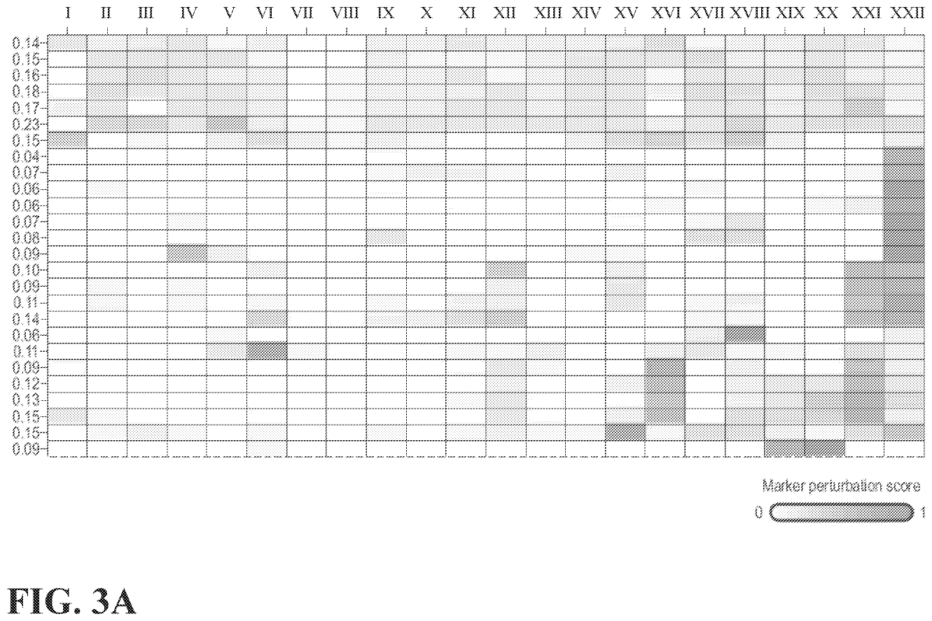

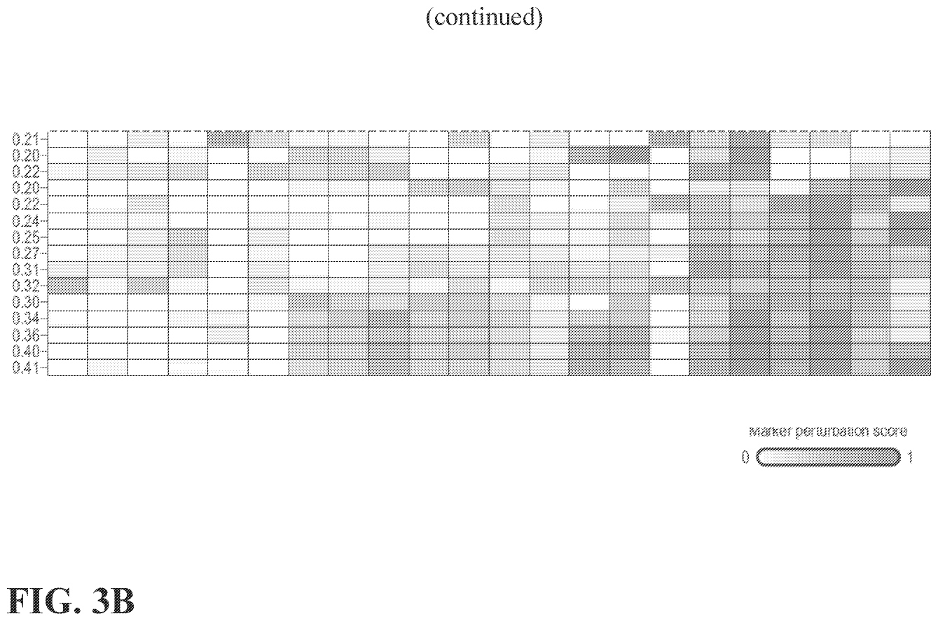

Marker perturbation scores and global condensate perturbation scores were visualized using Spotfire. As shown in FIGS. 3A and 3B, marker perturbation scores and global condensate perturbation scores for 40 candidate compounds at 3.33 μM concentration were assessed across 22 condensate markers (11 condensate types). As shown in FIGS. 3A and 3B, the different candidate compounds tested have very different condensate specificity as demonstrated by the wide range of global condensate perturbation scores, and the methods provided herein enable, e.g., identification of selective condensate modulators.

### Image Analysis Protocol Wherein the Global Condensate Perturbation Score is Based on Sums of Marker Perturbation Scores

Next, a modified Z-score for each feature assessed for each marker stained in each treatment condition tested was calculated. The result was an individual Z-score for each feature for each marker in each treatment condition that describe how much the feature differs compared to negative control treated wells. Z-scores were calculated using (Xᵢ-\tilde{X})/MAD, where Xᵢ was a single data value, \tilde{X} was the median of the negative control treated wells, and MAD was Median Absolute Deviation of the negative control treated wells. Marker perturbation scores were then calculated for each treatment condition for each marker stained. This score reflected how strongly a treatment condition perturbs a single condensate marker. Marker perturbation scores are determined by aggregating all the features that changed relative to negative control. First, each feature is given an individual feature-change score. Features with modified Z-scores with an absolute value <3 receive a feature-change score of 0. Features with modified Z-scores with an absolute value >10 receive a feature-change score of 1. Features with modified Z-scores with an absolute value >3 but <10 receive a feature-change score proportionally scaled between 0 and 1. The marker perturbation score is then calculated as the sum of all feature-change scores for the top 25% feature-change scores. For example, if 20 features are measured, the marker perturbation score would be calculated as the sum of the 5 highest feature-change scores.

Using the marker perturbation scores, a global condensate perturbation score was calculated for each treatment condition. This score reflected how broadly a treatment condition perturbs all condensates The global condensate perturbation score was calculated as the sum of all marker perturbation scores.

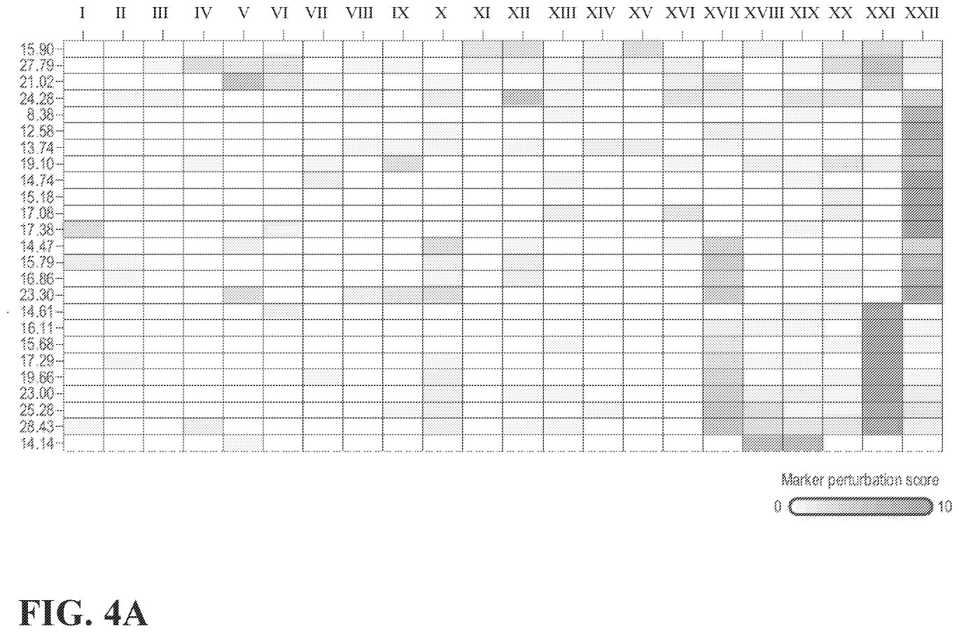

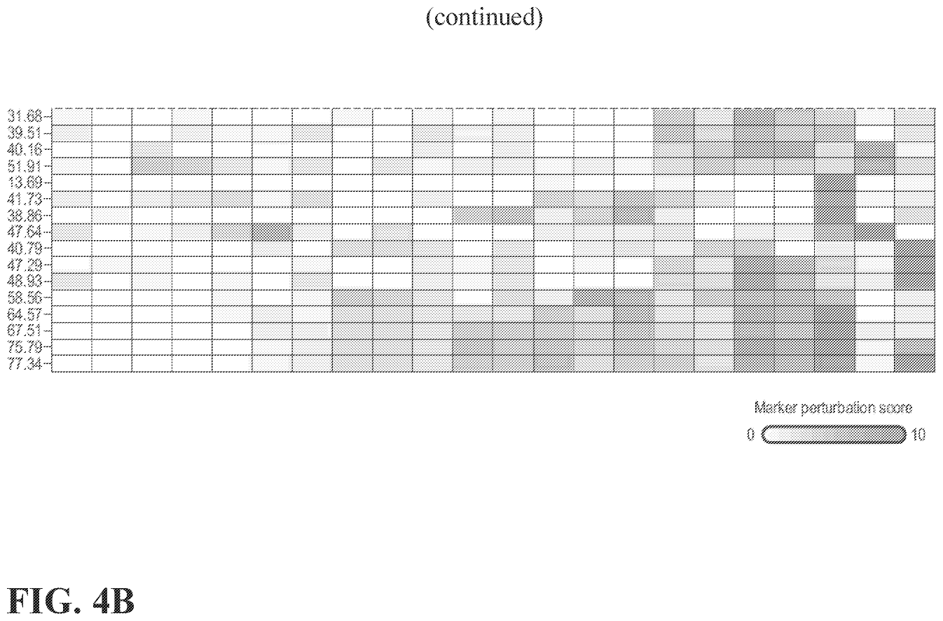

Marker perturbation scores and global condensate perturbation scores were visualized using Spotfire. As shown in FIGS. 4A and 4B, marker perturbation scores and global condensate perturbation scores for 40 candidate compounds at 3.33 μM concentration were assessed across 22 condensate markers (11 condensate types). As shown in FIGS. 4A and 4B, the different candidate compounds tested have very different condensate specificity as demonstrated by the wide range of global condensate perturbation scores, and the methods provided herein enable, e.g., identification of selective condensate modulators.

1. A method of identifying a stimulus for a condensate modulatory characteristic for one or more condensate types, the method comprising:
(a) subjecting a cellular composition comprising a cell type to the stimulus,
(b) measuring a feature of at least one marker in at least a portion of the stimulus-subjected cellular composition,
wherein the marker associates with at least one of the one or more condensate types prior to and/or after the cellular composition is subjected to the stimulus;
(c) determining a marker perturbation score for each marker in the stimulus-subjected cellular composition based on the measured feature of each marker; and
(d) identifying the stimulus for the condensate modulatory characteristic from the marker perturbation score.

2. The method of claim 1, further comprising the step of:
determining a global condensate perturbation score, wherein the global condensate perturbation score is based on at least one marker perturbation score; and
the identifying the stimulus for the condensate modulatory characteristic step can be identified from at least one marker perturbation score and/or global condensate perturbation score.

3. The method of claim 1, wherein the measuring the feature step measures two markers for each of the at least one or more condensate types in at least a portion of the stimulus-subjected cellular composition.

4. The method of claim 1, wherein the measuring the feature step measures at least one marker for at least two or more condensate types.

5. The method of claim 1, wherein each marker is independently a lipid, a polypeptide, or a nucleic acid.

6. The method of claim 1, wherein each marker is independently a polypeptide.

7. The method of claim 1, wherein each marker:
(i) is within the condensate type;
(ii) partitions into the condensate type after subjecting the cellular composition to the stimulus; or
(iii) is excluded from the condensate type after subjecting the cellular composition to the stimulus.

8. The method of claim 1, wherein each marker is independently a condensate scaffold polypeptide or nucleic acid.

9. The method of claim 8, wherein at least one marker is a condensate scaffold polypeptide.

10. The method of claim 1, wherein each marker is independently a condensate client polypeptide or nucleic acid.

11. The method of claim 1 wherein the measuring the feature of the at least one marker in at least a portion of the stimulus-subjected cellular composition comprises staining at least a portion of the cellular composition for the marker.

12. The method of claim 11, wherein the staining is immunofluorescent (IF) staining.

13. The method of claim 1, wherein the measuring, for at least the portion of the cellular composition, the feature of the at least one marker comprises imaging at least the portion of the cellular composition.

14. The method of claim 13, wherein the imaging comprises a fluorescent imaging technique.

15. The method of claim 1, wherein at least one of one or more different condensate types form in the cellular compositions after being subjected the stimulus.

16. The method of claim 1, wherein the stimulus is selected from the group consisting of an exogenous compound, exogenous peptidic material, exogenous genetic material, stressors, an environmental stimulus, and combinations thereof.

17. The method of claim 1, wherein the stimulus is an exogenous compound.

18. The method of claim 17, wherein the compound is a small molecule therapeutic candidate, or precursor thereof.

19. The method of claim 17, wherein the cellular composition is subjected to the compound at a known concentration.

20. The method of claim 1, further comprising subjecting the cellular composition to a second stimulus, measuring a feature of the at least one marker in at least a portion of the second stimulus-subjected cellular composition, and independently determining a marker perturbation score for each marker in the second stimulus-subjected cellular composition based on the measured feature of each marker in the second stimulus-subjected cellular composition.

21. The method of claim 20, further comprising comparing a marker perturbation score for a marker in the stimulus-subjected cellular composition with a marker perturbation score for a marker in the second stimulus-subjected cellular composition.

22. The method of claim 20, further comprising determining a second global condensate perturbation score to evaluate the second stimulus for the condensate modulatory characteristic, wherein the second global condensate perturbation score is based on at least one marker perturbation score for a marker in the second stimulus-subjected cellular composition.

23. The method of claim 22, further comprising comparing the global condensate perturbation score with the second global condensate perturbation score.

24. The method of claim 1, further comprising determining at least one characteristic associated with at least one of the one or more condensate types and/or the markers.

25. The method of claim 24, wherein the at least one characteristic associated with the at least one of the one or more condensate types and/or the markers comprises:
(i) location of the condensate type;
(ii) distribution of the condensate type and/or the marker;
(iii) number of the condensate type;
(iv) size of the condensate type;
(v) ratio of the amount of the condensate type and a control condensate;
(vi) a functional activity associated with the condensate type;
(vii) composition of the condensate type;
(viii) co-localization of the condensate type with a biomolecule;
(ix) diffusion coefficient of a component of the condensate type;
(x) stability of the condensate type;
(xi) dissolution or reduction in size of the condensate type;
(xii) surface area of the condensate type;
(xiii) sphericity of the condensate type;
(xiv) liquidity of the condensate type;
(xv) solidification of the condensate type;
(xvi) location of the marker;
(xvii) amount of the marker or a precursor thereof;
(xviii) condensate partitioning of the marker into the condensate type;
(xix) a functional activity associated with the marker;
(xx) aggregation of the marker;
(xxi) post-translational modification status of the marker; and
(xxii) amount of a degradation product of the marker.

26. The method of claim 1, wherein at least one of the one or more condensate types is selected from the group consisting of a cleavage body, p-granule, histone locus body, a multivesicular body, neuronal RNA granule, nuclear gem, nuclear pore complex, nuclear speckle, nuclear stress body, nucleolus, Oct1/PTF/transcription (OPT) domain, paraspeckle, perinucleolar compartment, PML nuclear body, PML oncogenic domain, polycomb body, processing body, Sam68 nuclear body, stress granule, splicing speckle, P62 body, Cajal body, heterochromatin, transcriptional condensate, and centrosome.

27. The method of claim 1, wherein the method comprises measuring a feature of a second marker in at least a portion of the stimulus-subjected cellular composition,
wherein the second marker associates with at least one of the one or more condensate types prior to and/or after the cellular composition is subjected to the stimulus.

28. The method of claim 27, further comprising determining a second marker perturbation score for the second marker in the stimulus-subjected cellular composition based on the measured feature of the second marker.

29. The method of claim 27, wherein the marker perturbation score is indicative of whether the stimulus modulates association of the marker with the at least one of the one or more condensate types in the cellular composition, and wherein the second marker perturbation score is indicative of whether the stimulus modulates association of the second marker with the at least one of the one or more condensate types in the cellular composition.

30. The method of claim 27, wherein the marker and the second marker associate with the same condensate type.

31. The method of claim 27, wherein the marker and the second marker associate with the different condensate types.

32. The method of claim 1, wherein each marker perturbation score is independently based on at least one characteristic associated with at least one of the one or more condensate types and/or the markers.

33. The method of claim 1, wherein each marker perturbation score is independently based on a coefficient of variation (CV) for the marker, and wherein the CV is determined based on a standard deviation (STD) of distribution intensity for the marker divided by the mean distribution intensity of the marker.

34. The method of claim 33, wherein the distribution intensity for each marker is based on a pixel analysis of one or more images of the one or more cellular compositions, or a portion of each thereof.

35. The method of claim 33, wherein each marker perturbation score is based on a median absolute deviation (MAD) Z-score of the CV for the marker.

36. The method of claim 35, further comprising obtaining the MAD Z-score for the marker.

37. The method of claim 35, wherein:
(i) the marker perturbation score is 1 when the MAD Z-score is >5 or <−5;
(ii) the marker perturbation score is 0.5 when 2.5<MAD Z-score≤5 or −5≤MAD Z-score<−2.5; and
(iii) the marker perturbation score is 0 when −2.5 ≤MAD Z-score≤2.5.

38. The method of claim 1, wherein the determining the marker perturbation score for a marker comprises extracting one or more features from an image of the stimulus-subject cellular composition.

39. The method of claim 38, wherein the one or more features are based on one or more of: one or more texture features, one or more intensity features, or one or more morphology features.

40. The method of claim 38, wherein the determining the marker perturbation score for a marker comprises measuring a plurality of features associated with the marker, calculating a modified Z-score for each feature of each marker based on the measured plurality of features, calculating a feature-change score for each feature of each marker based on the associated modified Z-score and a proportional scale, and then aggregating feature-change scores of a top percentage of the feature-change scores.

41. The method of claim 40, wherein the aggregating the modified Z-scores comprises summing or averaging.

42. The method of claim 1, further comprising evaluating a reference stimulus, wherein the method comprises determining a reference marker perturbation score.

43. The method of claim 42, wherein the reference marker perturbation score is 0, and wherein the marker perturbation score of >0 is indicative of the stimulus having a condensate modulatory characteristic.

44. The method of claim 2, wherein the global condensate perturbation score is indicative of the stimulus selectively modulating the one or more condensate types for the stimulus-subjected cellular composition.

45. The method of claim 2, wherein the global condensate perturbation score is indicative of the stimulus non-selectively modulating the one or more condensate types for the stimulus-treated cellular composition.

46. The method of claim 2, wherein the global condensate perturbation score is indicative of the stimulus not substantially modulating the one or more target condensate types for the stimulus-treated cellular composition.

47. The method of claim 28, wherein the global condensate perturbation score is based on the marker perturbation score and the second marker perturbation score.

48. The method of claim 2, wherein the global condensate perturbation score is calculated by dividing [a sum of all marker perturbation scores] with [the number of marker perturbation scores].

49. The method of claim 2, wherein the global condensate perturbation score is calculated by summing all marker perturbation scores.

50. The method of claim 2, wherein the method comprises determining a reference global condensate perturbation score.

51. The method of claim 50, wherein the reference global condensate perturbation score is 1, and wherein:
(i) the global condensate perturbation score of 0< and <1 is indicative of the stimulus selectively modulating the one or more condensate types in the cellular composition; and
(ii) the global condensate perturbation score of 0 is indicative of the stimulus not substantially modulating the one or more condensate types in the cellular composition.

52. The method of claim 51, wherein for the global condensate perturbation score of 0 <and <1, the smaller the global condensate perturbation score for the stimulus the higher the selectivity of the stimulus.
